SEQUENCE LISTING (1 sequence)

SEQ ID NO: 1 - DNA, 30 bp, Artificial Sequence
  Description of Artificial Sequence: Synthetic oligonucleotide
tcgaacgttcgaacgttcgaacgttcgaat

(19) United States
(12) Patent Application Publication
(10) Pub. No.: US 2023/0364125 A1
(43) Pub. Date: Nov. 16, 2023

(54) CANCER THERAPY USING TOLL-LIKE RECEPTOR AGONISTS

(71) Applicant: TriSalus Life Sciences, Inc., Westminster, CO (US)

(72) Inventors: Steven C. Katz, East Greenwich, RI (US); Bryan F. Cox, Spring Grove, IL (US); David Benjamin Jaroch, Arvada, CO (US)

(73) Assignee: TriSalus Life Sciences, Inc., Westminster, CO (US)

(21) Appl. No.: 18/027,883

(22) PCT Filed: Sep. 21, 2021
(86) PCT No.: PCT/US2021/051378

§ 371 Date: Mar. 22, 2023

Related U.S. Application Data
(60) Provisional application No. 63/225,026, filed on Jul. 23, 2021.
(60) Provisional application No. 63/159,867, filed on Mar. 11, 2021.
(60) Provisional application No. 63/159,857, filed on Mar. 11, 2021.
(60) Provisional application No. 63/139,622, filed on Jan. 20, 2021.
(60) Provisional application No. 63/115,435, filed on Nov. 18, 2020.
(60) Provisional application No. 63/081,613, filed on Sep. 22, 2020.

(51) Int. Cl. A61K 31/7125; A61K 9/00; A61P 35/04; A61K 45/06

(52) U.S. Cl. CPC A61K 31/7125; A61K 9/0019; A61K 45/06; A61P 35/04

(57) ABSTRACT

Embodiments of the present invention provide for methods of treating cancer and methods of delivering toll-like receptor (TLR) agonists to solid tumors in the liver using a locoregional therapy through the vasculature. In one aspect, the present invention relates to a method of treating metastases of uveal melanoma of the liver comprising administering TLR9 agonists to the liver.

### CROSS-REFERENCE TO RELATED APPLICATION(S)

This application claims the benefit of: U.S. Provisional Pat. Application No. 63/081,613, which was filed on Sep. 22, 2020; U.S. Provisional Pat. Application No. 63/115,435, which was filed on Nov. 18, 2020; U.S. Provisional Pat. Application No. 63/139,622, which was filed on Jan. 20, 2021; U.S. Provisional Pat. Application No. 63/159,857, which was filed on Mar. 11, 2021; U.S. Provisional Pat. Application No. 63/159,867, which was filed on Mar. 11, 2021; and U.S. Provisional Pat. Application No. 63/225,026, which was filed on Jul. 23, 2021, all of which are incorporated by reference in their entireties.

### SEQUENCE LISTING

The instant application contains a Sequence Listing which has been submitted electronically in ASCII format and is hereby incorporated by reference in its entirety. Said ASCII copy, created on Sep. 16, 2020, is named A372-502_SL.txt and is 484 bytes in size.

### FIELD OF THE INVENTION

The present disclosure relates generally to methods of treating cancer and methods of delivering toll-like receptor (TLR) agonists to solid tumors in the liver using a locoregional therapy through the vasculature.

### BACKGROUND OF THE INVENTION

Cancer is a devastating disease that involves the unchecked growth of cells, which may result in the growth of solid tumors in a variety of organs such as the skin and liver. Tumors may first present in any number of organs or may be the result of metastases or spread from other locations.

Melanoma is a diverse disease that encompasses a wide range of subtypes and presentation features. Melanoma is a cancer that develops from melanocytes, and may involve a number of subtypes and presentations, including in any organ or tissue where melanocytes are found, such as the skin and the eye, and also including metastases to other organs such as the liver.

Uveal melanoma (UM) is the most common primary intraocular malignancy accounting for 85-95% of primary ocular malignancies and 3-5% of all melanoma cases. These malignancies arise from melanocytes within the uveal tract, which consists of the iris, ciliary body, and choroid. Definitive treatment of the primary tumor with radiotherapy or enucleation results in low rates of local recurrence. However, despite effective local control, metastatic disease occurs in >50% of patients. Metastatic uveal melanoma typically involves the liver in >90% of cases and arises from hematologic spread. The disease’s proclivity for liver metastases (LM) has been evaluated in murine and human tumor studies. Other sites of involvement for metastatic uveal melanoma include the lung, bone, brain, lymph nodes and skin. The solid tumors present in the liver from metastatic uveal melanoma (e.g. multifocal visceral tumors) are particularly difficult to treat.

For metastatic disease, treatment can be grouped into several categories, including liver-directed therapies, cytotoxic chemotherapy, immunotherapy, molecular-targeted therapies, and epigenetic modifiers. Metastatic disease has very poor outcomes with 1-year overall survival of 43%, from the time of the original diagnosis.

Further, although certain antibody therapies such as ipilimumab, nivolumab, pembrolizumab, and atezolizumab have been approved for the treatment of cutaneous melanoma via intravenous systemic infusion, these therapies have not been approved as safe or efficacious for the treatment of metastatic uveal melanoma.

Accordingly, there remains a need for a safe and effective therapy for the treatment of metastatic uveal melanoma.

### SUMMARY OF THE INVENTION

The present invention relates to methods of treating cancer and methods of delivering TLR agonists to solid tumors in the liver using a locoregional therapy through the vasculature.

In one aspect, the present invention relates to a method of treating metastases of uveal melanoma of the liver comprising administering a TLR agonist through an intravascular device by hepatic arterial infusion (HAI). According to another embodiment, the treatment of the metastases of uveal melanoma of the liver comprises administering a TLR agonist through an intravascular device by portal vein infusion (PVI).

In some embodiments, the TLR agonists are administered through pressure-enabled drug delivery (PEDD), which includes the administration of a therapeutic through a device, such as a catheter device, which generates, causes, and/or contributes to a net increase in fluid pressure within the vessel and/or target tissue or tumor.

In some embodiments, the TLR agonists are administered through a pressure-enabled device, such as one that increases vascular pressure.

In some embodiments, the TLR agonist is a Class C type CpG oligodeoxynucleotide (CpG-C ODN).

In some embodiments, the administration of a TLR agonist through an intravascular device to the liver results in a reduction of myeloid-derived suppressor cells (MDSCs) or the functional alteration of MDSCs to limit immunosuppression.

In some embodiments, the TLR agonist is a TLR9 agonist.

These and other objects, features, and advantages of the exemplary embodiments of the present disclosure will become apparent upon reading the following detailed description of the exemplary embodiments of the present disclosure, when taken in conjunction with the entire specification.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure.

FIG. 1 illustrates the structure of SD-101.

FIGS. 2A-2B illustrate the effect of an exemplary combination of an exemplary TLR9 agonist and a checkpoint inhibitor on tumor progression in a murine model for liver metastases.

FIGS. 3A-3D illustrate the effect of exemplary TLR9 agonists on the MDSC population, PD-L1, and CD4 and CD8 population and activation expression in human PBMCs.

FIGS. 4A-4D illustrate the effect of exemplary TLR9 agonists on NFκB and IFNα regulated cytokine production in human PMBCs.

FIGS. 5A-5D illustrate the effect of an exemplary TLR9 agonists on human MDSC programming.

FIG. 6 illustrates the schema for developing liver metastases in mice and a corresponding treatment protocol for portal vein and tail vein administrations.

FIGS. 7A-7B illustrate the effect of an exemplary TLR9 agonist on tumor burden.

FIGS. 8A-8D illustrate a gating strategy and the effect of the exemplary TLR9 agonist on the MDSC population, M-MDSC population, and G-MDSC population.

FIGS. 9A-9C illustrate a gating strategy and the effect of the exemplary TLR9 agonist on the M1- and M2-macrophage populations in liver metastases.

FIGS. 10A-10B illustrate the effect of the exemplary TLR9 agonist on NFκB, STAT3 activation, and IL6 production.

FIGS. 11A-11B illustrate effect of the exemplary TLR9 agonist’s concentration on NFκB signal activity.

FIG. 12 illustrates a sample processing methodology of a liver four primary lobes, which are sectioned into 1 cm thick slices.

FIGS. 13A-13B illustrate representative histograms of pixel intensity values in untreated and treated tissue, respectively.

FIGS. 14A-14B illustrate near-IR imaging comparing delivery of a labeled ODN by needle injection (FIG. 14A) relative to delivery to local arterial network using a PEDD device (FIG. 14B).

FIGS. 15A-15B illustrate near-IR imaging comparing delivery of a labeled ODN by needle injection (FIG. 15A) relative to delivery to local arterial network using a PEDD device (FIG. 15B).

FIGS. 16A-16B illustrate signal intensity of labeled ODN 2395 and SD-101 individually, administered by needle and PEDD, as well as signal intensity for both compounds combined.

FIGS. 17A-17B illustrate therapeutic coverage for labeled ODN 2395 and SD-101 individually, administered by needle and PEDD, as well as tissue volume for both compounds combined.

FIG. 18 illustrates liver enzyme response after hepatic artery infusion with labelled oligonucleotide.

FIG. 19 illustrates signal intensity of labeled ODN retained in porcine liver tissue after delivery by an end-hole catheter or by PEDD.

FIGS. 20A-20C illustrate porcine liver tissue displaying a high degree of signal overlap with end-hole and PEDD devices.

FIGS. 21A-21C illustrate porcine liver tissue displaying a low degree of signal overlap with end-hole and PEDD devices.

FIG. 22 illustrates Venn diagram of end-hole mean treated tissue volume, PEDD mean treated tissue volume, and overlapping co-treated tissue volume.

FIG. 23 illustrates an overall clinical study design for treating uveal melanoma liver metastasis according to an embodiment of the invention.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to understand the different described aspects of the invention. The description of embodiments should facilitate understanding of the invention to such an extent that other implementations, not specifically covered but within the knowledge of a person of skill in the art having read the description of embodiments, would be understood to be consistent with application of the invention.

### Toll-Like Receptor Agonists

Toll-like receptors are pattern recognition receptors that can detect microbial pathogen-associated molecular patterns (PAMPs). TLR stimulation, such as TLR9 stimulation, can not only provide broad innate immune stimulation, but can also specifically address the dominant drivers of immunosuppression in the liver. TLR1-10 are expressed in humans and recognize a diverse variety of microbial PAMPs. In this regard, TLR9 can respond to unmethylated CpG-DNA, including microbial DNA. CpG refers to the motif of a cytosine and guanine dinucleotide 1. TLR9 is constitutively expressed in B cells, plasmacytoid dendritic cells (pDCs), activated neutrophils, monocytes/macrophages, T cells, and MDSCs. TLR9 is also expressed in non-immune cells, including keratinocytes and gut, cervical, and respiratory epithelial cells. TLR9 can bind to its agonists within endosomes. Signaling may be carried out through MYD88/IkB/NfκB to induce pro-inflammatory cytokine gene expression. A parallel signaling pathway through IRF7 induces type 1 and 2 interferons (e.g. IFN-α, IFN-γ, etc.) which stimulate adaptive immune responses. Further, TLR9 agonists can induce cytokine and IFN production and functional maturation of antigen presenting dendritic cells.

According to an embodiment, a TLR9 agonist can reduce and reprogram MDSCs. MDSCs are key drivers of immunosuppression in the liver. MDSCs also drive expansion of other suppressor cell types such as T regulatory cells (Tregs), tumor-associated macrophages (TAMs), and cancer-associated fibroblasts (CAFs). MDSCs may downregulate immune cells and interfere with the effectiveness of immunotherapeutics. Further, high MDSC levels generally predict poor outcomes in cancer patients. In this regard, reducing, altering, or eliminating MDSCs is thought to improve the ability of the host’s immune system to attack the cancer as well as the ability of the immunotherapy to induce more beneficial therapeutic responses. In an embodiment, TLR9 agonists may convert MDSCs into immunostimulatory M1 macrophages, convert immature dendritic cells to mature dendritic cells, and expand effector T cells to create a responsive tumor microenvironment to promote anti-tumor activity.

According to an embodiment, synthetic CpG-oligonucleotides (CPG-ONs) mimicking the immunostimulatory nature of microbial CpG-DNA can be developed for therapeutic use. According to an embodiment, the oligonucleotide is an oligodeoxynucleotide (ODN). There are a number of different CpG-ODN class types, e.g. Class A, Class B, Class C, Class P, and Class S, which share certain structural and functional features. In this regard, Class A type CPG-ODNs (or CPG-A ODNs) are associated with pDC maturation with little effect on B cells as well as the highest degree of IFNα induction; Class B type CPG-ODNs (or CPG-B ODNs) strongly induce B-cell proliferation, activate pDC and monocyte maturation, NK cell activation, and inflammatory cytokine production; and Class C type CPG-ODNs (or CPG-C ODNs) can induce B-cell proliferation and IFN-α production. Further, according to an embodiment, CPG-C ODNs can be associated with the following attributes: (i) unmethylated dinucleotide CpG motifs, (ii) juxtaposed CpG motifs with flanking nucleotides (e.g. AACGTTCGAA), (iii) a complete phosphorothioate (PS) backbone that links the nucleotides (as opposed to the natural phosphodiester (PO) backbones found in bacterial DNA), and (iv) a self-complimentary, palindromic sequence (e.g. AACGTT). In this regard, CPG-C ODNs may bind themselves due to their palindromic nature, thereby producing double-stranded duplex or hairpin structures.

Further, according to an embodiment, the CPG-C ODNs can include one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In an embodiment, the CpG-C ODNs are 12 to 100 bases in length, preferably 12 to 50 bases in length, preferably 12 to 40 bases in length, or preferably 12-30 bases in length. In an embodiment, the CpG-C ODN is 30 bases in length. In an embodiment, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In an embodiment, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length.

In an embodiment, the at least one palindromic sequence is 8 to 97 bases in length, preferably 8 to 50 bases in length, or preferably 8 to 32 bases in length. In an embodiment, the at least one palindromic sequence is at least (lower limit) 8, 10, 12, 14, 16, 18, 20, 22, 24, 26, 28, or 30 bases in length. In an embodiment, the at least one palindromic sequence is at most (upper limit) 50, 48, 46, 44, 42, 40, 38, 36, 34, 32, 30, 28, 26, 24, 22, 20, 18, 16, 14, 12 or 10 bases in length.

In an embodiment, the CpG-C ODN can comprise the sequence of SEQ ID NO: 1.

According to an embodiment, the CpG-C ODN can comprise the SD-101. SD-101 is a 30-mer phosphorothioate oligodeoxynucleotide, having the following sequence:

5'-TCG AAC GTT CGA ACG TTC GAA CGT TCG AAT-3' (SEQ ID NO: 1)

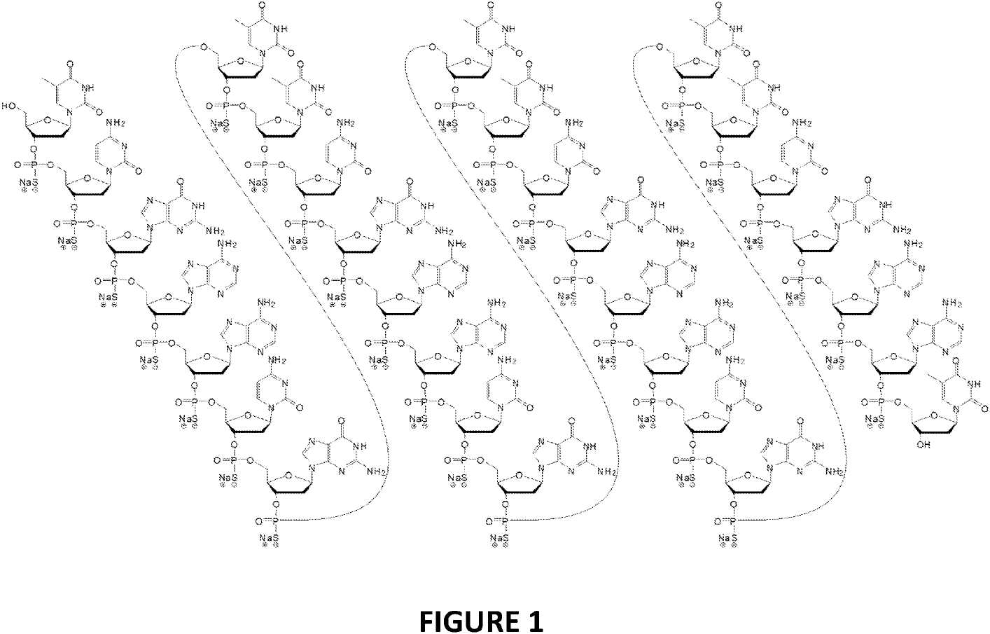

SD-101 drug substance is isolated as the sodium salt. The structure of SD-101 is illustrated in FIG. 1.

The molecular formula of SD-101 free acid is C₂₉₃ H₃₆₉ N₁₁₂ O₁₄₉ P₂₉ S₂₉ and the molecular mass of the SD-101 free acid is 9.672 Daltons. The molecular formula of SD-101 sodium salt is C₂₉₃ H₃₄₀ N₁₁₂ O₁₄₉ P₂₉ S₂₉ Na₂₉ and the molecular mass of the SD-101 sodium salt is 10,309 Daltons.

Further, according to an embodiment, the CPG-C ODN sequence can correspond to SEQ ID NO 172 as described in U.S. Pat. No. 9,422,564, which is incorporated by reference herein in its entirety.

In an embodiment, the CpG-C ODN can comprise a sequence that has at least 75% homology to any of the foregoing, such as SEQ ID NO: 1.

According to another embodiment the CPG-C ODN sequence can correspond to any one of the other sequences described in U.S. Pat. No. 9,422,564. Further, the CPG-C ODN sequence can also correspond to any of the sequences described in U.S. Pat. No. 8,372,413, which is also incorporated by reference herein in its entirety.

According to an embodiment, any of the CPG-C ODNs discussed herein may be present in their pharmaceutically acceptable salt forms. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In an embodiment, the CpG-C ODNs are in the ammonium, sodium, lithium, or potassium salt forms. In one preferred embodiment, the CpG-C ODNs are in the sodium salt form. The CpG-C ODN may be provided in a pharmaceutical solution comprising a pharmaceutically acceptable excipient. Alternatively, the CpG-C ODN may be provided as a lyophilized solid, which is subsequently reconstituted in sterile water, saline or a pharmaceutically acceptable buffer before administration. Pharmaceutically acceptable excipients of the present disclosure include for instance, solvents, bulking agents, buffering agents, tonicity adjusting agents, and preservatives. In an embodiment, the pharmaceutical compositions may comprise an excipient that functions as one or more of a solvent, a bulking agent, a buffering agent, and a tonicity adjusting agent (e.g. sodium chloride in saline may serve as both an aqueous vehicle and a tonicity adjusting agent). The pharmaceutical compositions of the present disclosure are suitable for parenteral and/or percutaneous administration.

In an embodiment, the pharmaceutical compositions comprise an aqueous vehicle as a solvent. Suitable vehicles include for instance sterile water, saline solution, phosphate buffered saline, and Ringer’s solution. In an embodiment, the composition is isotonic.

The pharmaceutical compositions may comprise a bulking agent. Bulking agents are particularly useful when the pharmaceutical composition is to be lyophilized before administration. In an embodiment, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In an embodiment, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin, and mannitol.

The pharmaceutical compositions may comprise a preservative. Suitable preservatives include for instance antioxidants and antimicrobial agents. However, in an embodiment, the pharmaceutical composition is prepared under sterile conditions and is in a single use container, and thus does not necessitate inclusion of a preservative.

Table 1 describes the batch formula for SD-101 Drug Product - 16 g/L:

TABLE 1

| Ingredient | Grade | Concentration | Amount per batch | Clinical Lot DVXA05 |
|---|---|---|---|---|
| | | | | Batch Size 2.224 L |
| SD-101 Drug Substance* | GMP | 1.6% | 16.00 g | 35.584 g¹ |
| Sodium phosphate, dibasic anhydrous | USP/NF, EP | 1.02% | 1.02 g | 2.268 g |
| Sodium phosphate, monobasic anhydrous | USP | 0.34% | 0.34 g | 0.747 g |
| Sodium chloride | USP/NF, EP | 7.31% | 7.31 g | 16.257 g |
| Sterile Water for Injection | USP/NF, EP | QS | QS | 2.224 L (kg) (QS) |

¹Quantity based upon measured content in solution (to exclude moisture present in lyophilized powder)
* SD-101 Drug Substance in Table 1 reflects the totality of all oligonucleotide content, including SD-101.

In some embodiments, the unit dose strength may include from about 0.1 mg/mL to about 20 mg/mL. In one embodiment, the unit dose strength of SD-101 is 13.4 mg/mL.

CpG-C ODNs may contain modifications. Suitable modifications can include but are not limited to, modifications of the 3′OH or 5′OH group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g. the palindromic portion of the CpG-C ODN remains self-complementary).

CpG-C ODNs may be linear, may be circular or include circular portions and/or a hairpin loop. CpG-C ODNs may be single stranded or double stranded. CpG-C ODNs may be DNA, RNA or a DNA/RNA hybrid.

CpG-C ODNs may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In an embodiment, CpG-C ODNs have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include but are not limited to addition of an electron-withdrawing moiety to C-5 and/or C-6 of a cytosine of the CpG-C ODN (e.g. 5-bromocytosine, 5-chlorocytosine, 5-fluorocytosine, 5-iodocytosine) and C-5 and/or C-6 of a uracil of the CpG-C ODN (e.g. 5-bromouracil, 5-chlorouracil, 5-fluorouracil, 5-iodouracil). As noted above, use of a base modification in a palindromic sequence of a CpG-C ODN should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e. double stranded) and hairpin forms of most ODNs are often in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked ODNs that are conformationally homogeneous and are “locked” in their most active form (either duplex or hairpin form) could potentially be more active than their uncrosslinked counterparts. Accordingly, some CpG-C ODNs of the present disclosure can contain covalent interstrand and/or intrastrand cross-links.

The techniques for making polynucleotides and modified polynucleotides are known in the art. Naturally occurring DNA or RNA, containing phosphodiester linkages, may be generally synthesized by sequentially coupling the appropriate nucleoside phosphoramidite to the 5′-hydroxy group of the growing ODN attached to a solid support at the 3′-end, followed by oxidation of the intermediate phosphite triester to a phosphate triester. Using this method, once the desired polynucleotide sequence has been synthesized, the polynucleotide is removed from the support, the phosphate triester groups are deprotected to phosphate diesters and the nucleoside bases are deprotected using aqueous ammonia or other bases.

The CpG-C ODN may contain phosphate-modified oligonucleotides, some of which are known to stabilize the ODN. Accordingly, some embodiments include stabilized CpG-C ODNs. The phosphorous derivative (or modified phosphate group), which can be attached to the sugar or sugar analog moiety in the ODN, can be a monophosphate, diphosphate, triphosphate, alkylphosphonate, phosphorothioate, phosphorodithioate, phosphoramidate or the like.

CpG-C ODNs can comprise one or more ribonucleotides (containing ribose as the only or principal sugar component), deoxyribonucleotides (containing deoxyribose as the principal sugar component), modified sugars or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is preferably the furanoside of ribose, deoxyribose, arabinose or 2′-0-alkylribose, and the sugar can be attached to the respective heterocyclic bases in either anomeric configuration. The preparation of these sugars or sugar analogs and the respective nucleosides wherein such sugars or analogs are attached to a heterocyclic base (nucleic acid base) per se is known, and therefore need not be described here. Sugar modifications may also be made and combined with any phosphate modification in the preparation of a CpG-C ODN.

The heterocyclic bases, or nucleic acid bases, which are incorporated in the CpG-C ODN can be the naturally-occurring principal purine and pyrimidine bases, (namely uracil, thymine, cytosine, adenine and guanine, as mentioned above), as well as naturally-occurring and synthetic modifications of said principal bases. Thus, a CpG-C ODN may include one or more of inosine, 2′-deoxyuridine, and 2-amino-2′-deoxyadenosine.

According to another embodiment, the CPG-ODN is one of a Class A type CPG-ODNs (CPGP-A ODNs), a Class B type CPG-ODNs (CPG-B ODNs), a Class P type CPG-ODNs (CPG-P ODN), and a Class S type CPG-ODNs (CPG-S ODN). In this regard, the CPG-A ODN can be CMP-001.

In another embodiment, the CPG-ODN can be tilsotolimod (IMO-2125).

### Checkpoint Inhibitors

According to an embodiment, the TLR agonists of the present invention may be used in combination with a checkpoint inhibitor (CPI). The checkpoint inhibitor can include a Programmed Death 1 receptor (PD-1) antagonist. A PD-1 antagonist can be any chemical compound or biological molecule that blocks binding of Programmed Cell Death 1 Ligand 1 (PD-L1) expressed on a cancer cell to PD-1 expressed on an immune cell (T cell, B cell or NKT cell) and preferably also blocks binding of PD-L2 Programmed Cell Death 1 Ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1.

According to an embodiment, the PD-1 antagonist can include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

According to an embodiment, the PD-1 antagonist can include an immunoadhesin that specifically binds to PD-1 or PD-L1, and preferably specifically binds to human PD-1 or human PD-L1, e.g. a fusion protein containing the extracellular or PD-1 binding portion of PD-L1 or PD-L2 fused to a constant region such as an Fc region of an immunoglobulin molecule.

According to an embodiment, the PD-1 antagonist can inhibit the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain.

According to an embodiment, the PD-1 antagonist can be one of nivolumab, pembrolizumab, and cemiplimab. According to another embodiment, nivolumab is administered intravenously (IV) via a peripheral vein at a dose of 480 mg every four weeks (“Q4W”). According to another embodiment, nivolumab is administered intravenously (IV) via a peripheral vein at a dose of nivolumab 1 mg/kg every three weeks (“Q3W”). In yet another embodiment, nivolumab is administered concomitantly, at the same time, at about the same time, or on the same day with SD-101. In another embodiment, nivolumab is administered one a weekly, every other week, every three weeks, every four weeks, or on a monthly basis following the administration of one or more cycles of SD-101.

According to another embodiment, the CPI can include a PD-L1 antagonist. In this regard, the PD-L1 antagonist can be one of atezolizumab, avelumab, and durvalumab.

According to another embodiment, the CPI can include a CTLA-4 antagonist. In this regard, the CTLA-4 antagonist can be ipilimumab. According to another embodiment, ipilimumab is administered intravenously (IV) via a peripheral vein at a dose of 3 mg/kg every three weeks. In yet another embodiment, ipilimumab is administered concomitantly, at the same time, at about the same time, or on the same day with SD-101. In another embodiment, nivolumab is administered once a week, every other week, every three weeks, every four weeks, or on a monthly basis following the administration of one or more cycles of SD-101.

### Devices to Achieve Locoregional Delivery

According to an embodiment, any of the above-described devices may comprise any device useful to achieve locoregional delivery to a tumor, including a catheter itself, or may comprise a catheter along with other components (e.g. filter valve, balloon, pressure sensor system, pump system, syringe, outer delivery catheter, etc.) that may be used in combination with the catheter. In certain embodiments, the catheter is a microcatheter.

In some embodiments, the device may have one or more attributes that include, but are not limited to, self-centering capability that can provide homogeneous distribution of therapy in downstream branching network of vessels; anti-reflux capability that can block or inhibit the retrograde flow of the TLR agonist (for example, with the use of a valve and filter, and/or balloon); a system to measure the pressure inside the vessel; and a means to modulate the pressure inside the vessel, such as by causing a decrease in pressure at placement and during the 2 cc/min infusion, and an increase of pressure during saline bolus. In some embodiments, the system is designed to continuously monitor real-time pressure throughout the procedure.

In some embodiments, the device that may be used to perform the methods of the present invention is a device as disclosed in U.S. Pat. No. 8,500,775, U.S. Pat. No. 8,696,698, U.S. Pat. No. 8,696,699, U.S. Pat. No. 9,539,081, U.S. Pat. No. 9,808,332, U.S. Pat. No. 9,770,319, U.S. Pat. No. 9,968,740, U.S. Pat. No. 10,813,739, U.S. Pat. No. 10,588,636, U.S. Pat. No. 11,090,460,U.S. Pat. Publication No. 2018/0193591, U.S. Pat. Publication No. 2018/0250469, U.S. Pat. Publication No. 2019/0298983, U.S. Pat. Publication No. 2020/0038586, and U.S. Pat. Publication No. 2020-0383688, which are all incorporated by reference herein in their entireties.

In some embodiments, the device is a device as disclosed in U.S. Pat. No. 9,770,319. In certain embodiments, the device may be a device known as the Surefire Infusion System.

In some embodiments, the device supports the measurement of intravascular pressure during use. In some embodiments, the device is a device as disclosed in U.S. Pat. Publication No. 2020-0383688. In certain embodiments, the device may be a device known as the TriSalus Infusion System. In certain embodiments, the device may be a device known as the TriNav® Infusion System. The TriNav® is a single lumen catheter equipped with a one-way valve that responds dynamically to local pressure changes, such as those arising from the cardiac cycle or generated by infusion. The valve structure modulates distal vascular pressures and blood flow. This in turn may alter therapeutic distribution and first-pass absorption due to increased contact time within the vasculature.

In some embodiments, the TLR agonist may be administered through a device via PEDD. In some embodiments, the TLR agonist may be administered while monitoring the pressure in the vessel, which can be used to adjust and correct the positioning of the device at the infusion site and/or to adjust the rate of infusion. Pressure may be monitored by, for example, a pressure sensor system comprising one or more pressure sensors.

The rate of infusion may be adjusted to alter vascular pressure, which may promote the penetration of the TLR agonist into the target tissue or tumor. In some embodiments, the rate of infusion may be adjusted and/or controlled using a syringe pump as part of the delivery system. In some embodiments, the rate of infusion may be adjusted and/or controlled using a pump system. In some embodiments, the rate of infusion using a pump system may be about 0.1 cc/min to about 40 cc/min, or about 0.1 cc/min to about 30 cc/min, or about 0.5 cc/min to about 25 cc/min, or about 0.5 cc/min to about 20 cc/min, or about 1 cc/min to about 15 cc/min, or about 1 cc/min to about 10 cc/min, or about 1 cc/min to about 8 cc/min, or about 1 cc/min to about 5 cc/min. Further, the rate of infusion using a bolus infusion may be about 30 cc/min to about 360 cc/min, or about 120 cc/min to about 240 cc/min. In one embodiment the SD-101 infusion procedure lasts approximately 30-60 minutes. In an additional embodiment, SD-101 is administered for a period of time about 25 minutes.

### Methods Comprising Administration to the Liver

In an embodiment, the methods of the present invention include methods of treating a solid tumor in the liver, such as a tumor that is the metastasis of a melanoma, such as uveal melanoma, said method comprising administering a toll-like receptor agonist to a patient in need thereof, wherein the toll-like receptor agonist is administered through a device by HAI to such solid tumor in the liver. HAI refers to the infusion of a treatment into the hepatic artery of the liver. According to an embodiment, the toll-like receptor agonist or agonists are introduced through the percutaneous introduction of a device into the branches of a hepatic artery, such as a catheter and/or a device that facilitates pressure-enabled delivery. According to an embodiment, the toll-like receptor agonist is a TLR9 agonist and in some embodiments the TLR9 agonist is SD-101. In one embodiment, the patient is a human patient.

According to another embodiment, the methods of the present invention include methods of treating a solid tumor in the liver, such as a tumor that is the metastasis of a melanoma, such as uveal melanoma, said method comprising administering a toll-like receptor agonist to a patient in need thereof, wherein the toll-like receptor agonist is administered through a device by PVI to such solid tumor in the liver. PVI refers to the infusion of a treatment into the hepatic portal venous system. According to an embodiment, the toll-like receptor agonist or agonists are introduced through the percutaneous introduction of a device into the branches of the hepatic portal venous system, such as a catheter and/or a device that facilitates pressure-enabled delivery. According to an embodiment, the toll-like receptor agonist is a TLR9 agonist and in some embodiments the TLR9 agonist is SD-101. In one embodiment, the patient is a human patient.

According to one embodiment, the methods of the present invention include a method for treating a liver metastasis of uveal melanoma, wherein the subject has histologically or cytologically confirmed metastatic UM with liver-only disease or liver-dominant disease. Liver-dominant disease may present with intrahepatic metastases representing the largest fraction of disease relative to other organs, with permissible extrahepatic sites being the lungs, skin or subcutaneous tissues, and bone. Liver-dominant disease may also present with intrahepatic metastases representing the largest fraction of disease relative to other organs, or if progression of LM represents a significant threat to the patient’s life. According to another embodiment, the methods include administration to a subject who is male or female, and is eighteen years of age or older.

According to another embodiment, the methods of the present invention include a method for treating a liver metastasis of uveal melanoma, wherein the subject has not received prior cytotoxic chemotherapy, targeted therapy, or external radiation therapy within 14 days prior to enrollment. According to another embodiment, the methods of the present invention include administration to subjects who have not received therapy with prior immunological checkpoint blockade within 30 days before the first dose of study intervention and have no ongoing immune-mediated AEs Grade 2 or higher. According to yet another embodiment, methods of the present invention are administered to subject who have not ever received therapy with prior immunological checkpoint blockade. According to an embodiment of the invention, methods also include administration to subject who have not ever received prior embolic HAI therapy with permanent embolic material. In another embodiment, methods of the present invention include subjects who have had prior surgical resection or radiofrequency ablation of oligometastatic liver disease.

According to another embodiment, methods of the present invention include a method for treating a liver metastasis of uveal melanoma, wherein the subject has no prior history of or other concurrent malignancy unless the malignancy is clinically insignificant. In another embodiment, subjects who are treated according to the methods of the present invention may have no ongoing treatment. In yet another embodiment, the subject is clinically stable.

In another embodiment, methods of the present invention may include administration to a subject who has measurable disease in the liver according to RECIST v.1.1 criteria. In an additional embodiment, methods of the present invention may include administration to a subject who exhibits an Eastern Cooperative Oncology Group (“ECOG”) performance score (“PS”) of 0-1 at screening. In another embodiment, subjects who are administered therapy according to methods of the present invention have a life expectancy of greater than 3 months at screening as estimated by the investigator. In yet another embodiment, subjects have a QTc interval ≤480 msec.

In another embodiment, all associated clinically significant drug-related toxicity from previous cancer therapy is resolved prior to treatment. In this embodiment, resolution is to Grade ≤1 or the patient’s pretreatment level. In an additional embodiment, the subject may have Grade 2 alopecia and endocrinopathies controlled on replacement therapy.

In another embodiment, methods of the present invention may include administration to a subject who has adequate organ function at screening. In an embodiment, a subject with adequate organ function may exhibit one or more of the following: (i) platelet count >100,000/µL, (2) hemoglobin ≥8.0 g/dL, (3) white blood cell count (WBC) >2,000/µL (4) Serum creatinine ≤2.0 mg/dL unless the measured creatinine clearance is >40 mL/min/1.73 m², (5) total and direct bilirubin ≤2.0 × the upper limit of normal (ULN) and alkaline phosphatase ≤5 × ULN, (6) for patients with documented Gilbert’s disease, total bilirubin up to 3.0 mg/dL, (7) ALT and AST ≤5 × ULN, and (8) prothrombin time/International Normalized Ratio (INR) or activated partial thromboplastin time (aPTT) test results at screening ≤1.5 × ULN (this applies only to patients who do not receive therapeutic anticoagulation; patients receiving therapeutic anticoagulation should be on a stable dose for at least 4 weeks prior to the first dose of study intervention).

According to another embodiment, the methods of the present invention can also be used to treat liver metastases as a result of other cancers, e.g. colorectal cancer (i.e. colorectal cancer liver metastases), pancreatic cancers such as pancreatic ductal adenocarcinoma (i.e. pancreatic ductal adenocarcinoma liver metastases).

According to another embodiment, the tumor is unresectable.

According to another embodiment, the methods of the present invention can be administered with other cancer therapeutics such as immuno-modulators, tumor-killing agents, and/or other targeted therapeutics.

According to an embodiment, TLR9 therapy may be administered in combination with cell therapy, thereby enabling cell therapy by modulation of the immune system.

In one embodiment, the above methods of administration to the liver are intended to result in the penetration of the toll-like receptor agonist throughout the solid tumor, throughout the entire organ, or substantially throughout the entire tumor. In an embodiment, such methods enhance perfusion of the toll-like receptor agonist to a patient in need thereof, including by overcoming interstitial fluid pressure and solid stress of the tumor. In another embodiment, perfusion throughout an entire organ or portion thereof, may provide benefits for the treatment of the disease by thoroughly exposing the tumor to therapeutic agent. In an embodiment, such methods are better able to afford delivery of the toll-like receptor to areas of the tumor that have poor access to systemic circulation. In another embodiment, such methods deliver higher concentrations of the toll-like receptor agonist into such a tumor with less toll-like receptor agonist delivered to non-target tissues compared to conventional systemic delivery via a peripheral vein, or via direct intertumoral injection. In one embodiment, such methods result in the reduction in size, reduction in growth rate, or shrinkage or elimination of the solid tumor.

The methods of the present invention may also include mapping the vessels leading to the right and left lobes of the liver prior to performing HAI, or selective infusion into specific sectors or segments, and when necessary, occluding vessels that do not lead to the liver or that are otherwise not a target. In some embodiments, prior to infusion, patients can undergo a mapping angiogram, e.g. via a common femoral artery approach.

Methods for mapping vessels in the body and delivery of therapeutics are well known to the ordinarily skilled artisan. Occlusion may be achieved, for example, through the use of microcoil embolization, which allows the practitioner to block off-target arteries or vessels, thereby optimizing delivery of the modified cells to the liver. Microcoil embolization can be performed as needed, such as prior to administering the first dose of TLR9 agonists to facilitate optimal infusion of a pharmaceutical composition comprising the TLR9 agonists. In another embodiment, a sterile sponge (e.g. GELFOAM) can be used. In this regard, the sterile sponge can be cut and pushed into the catheter. In another embodiment, the sterile sponge can be provided as granules.

In some embodiments, doses of a TLR9 agonist, such as SD-101 may be about 0.01 mg, about 0.03 mg, about 0.05 mg, about 0.1 mg, about 0.3 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, or about 8 mg. In some embodiments, SD-101 is administered at doses of 12 mg, 16 mg, and 20 mg. Administration of a milligram amount of SD-101 (e.g. about 2 mg) describes administering about 2 mg of the composition illustrated in FIG. 1. For example, such an amount of SD-101 (e.g. about a 2 mg amount) may also exist within a composition that contains material in addition to such amount of SD-101, such as other related and unrelated compounds. Equivalent molar amounts of other pharmaceutically acceptable salts are also contemplated.

In some embodiments, doses of a TLR9 agonist, such as SD-101 may be between about 0.01 mg and about 10 mg, between about 0.01 mg and about 8 mg, and between about 0.01 mg and about 4 mg. In some embodiments, doses of a TLR9 agonist, such as SD-101 may be between about 2 mg and about 10 mg, between about 2 mg and about 8 mg, and between about 2 mg and about 4 mg. In some embodiments, doses of a TLR9 agonist, such as SD-101 may be less than about 10 mg, less than about 8 mg, less than about 4 mg, or less than about 2 mg. Such doses may be administered daily, weekly, or every other week. In one embodiment, doses of SD-101 are incrementally increased, such as through administration of about 2 mg, followed by about 4 mg, and then followed by about 8 mg.

In some embodiments, the methods of the present invention may comprise administering a dosing regimen comprising cycles, in which one or more of the cycles comprise administering SD-101 via HAI and PEDD. As used herein, a “cycle” is a repeat of a dosing sequence. In one embodiment, one cycle comprises three weekly doses per cycle (i.e. administration of SD-101 once per week over three consecutive weeks). In one embodiment, a cycle of treatment according to the present invention may comprise periods of SD-101 administration followed by “off” periods or rest periods. In another embodiment, in addition to three weekly doses per cycle, the cycle further comprises one week, two weeks, three weeks, or four weeks as a rest period following the weekly administration of SD-101. In yet another embodiment, in addition to three weekly doses per cycle, the cycle further comprises about thirty-eight days as a rest period following the weekly administration of SD-101. In another embodiment, the entire cycle comprises about fifty-two days. In another embodiment, the dosing regimen comprises at least one, at least two, or at least three cycles, or longer.

In some embodiments, the present invention relates to the use of a TLR9 agonist in the manufacture of a medicament for treating a solid tumor in the liver, such as a tumor that is the metastasis of a melanoma, such as uveal melanoma, said method comprising administering the TLR9 agonist to a patient in need thereof, wherein the TLR9 agonist is administered through a device by HAI to such solid tumor in the liver.

In some embodiments, SD-101 is administered for the treatment of metastatic uveal melanoma at a dose of 0.5 mg through HAI, and in some embodiments, the SD-101 is further administered through a device that modulates pressure (i.e. PEDD). In some embodiments, SD-101 is administered at a dose of 0.5 mg through HAI through a device that modulates vascular pressure in combination with a checkpoint inhibitor, wherein the checkpoint inhibitor is nivolumab. In other embodiments, SD-101 is administered at a dose of 0.5 mg through HAI and through a device that modulates pressure in combination with ipilimumab. In some embodiments, SD-101 is administered at a dose of 0.5 mg through HAI and through a device that modulates pressure in combination with ipilimumab and nivolumab.

In some embodiments, SD-101 is administered for the treatment of metastatic uveal melanoma at a dose of 2 mg through HAI, and in some embodiments, the SD-101 is further administered through a device that modulates pressure (i.e. PEDD). In some embodiments, SD-101 is administered at a dose of 2 mg through HAI through a device that modulates vascular pressure in combination with a checkpoint inhibitor, wherein the checkpoint inhibitor is nivolumab. In other embodiments, SD-101 is administered at a dose of 2 mg through HAI and through a device that modulates pressure in combination with ipilimumab. In some embodiments, SD-101 is administered at a dose of 2 mg through HAI and through a device that modulates pressure in combination with ipilimumab and nivolumab.

In some embodiments, SD-101 is administered for the treatment of metastatic uveal melanoma at a dose of 4 mg through HAI, and in some embodiments, the SD-101 is further administered through a device that modulates pressure (i.e. PEDD). In some embodiments, SD-101 is administered at a dose of 4 mg through HAI through a device that modulates vascular pressure in combination with a checkpoint inhibitor, wherein the checkpoint inhibitor is nivolumab. In other embodiments, SD-101 is administered at a dose of 4 mg through HAI and through a device that modulates pressure in combination with ipilimumab. In some embodiments, SD-101 is administered at a dose of 4 mg through HAI and through a device that modulates pressure in combination with ipilimumab and nivolumab.

In some embodiments, SD-101 is administered for the treatment of metastatic uveal melanoma at a dose of 8 mg through HAI, and in some embodiments, the SD-101 is further administered through a device that modulates pressure (i.e. PEDD). In some embodiments, SD-101 is administered at a dose of 8 mg through HAI through a device that modulates vascular pressure in combination with a checkpoint inhibitor, wherein the checkpoint inhibitor is nivolumab. In other embodiments, SD-101 is administered at a dose of 8 mg through HAI and through a device that modulates pressure in combination with ipilimumab. In some embodiments, SD-101 is administered at a dose of 8 mg through HAI and through a device that modulates pressure in combination with ipilimumab and nivolumab.

In some embodiments, the methods of the present invention result in the treatment of target lesions. In this embodiment, the methods of the present invention may result in a complete response, comprising the disappearance of all target lesions. In some embodiments, the methods of the present invention may result in a partial response, comprising at least a 30% decrease in the sum of the longest diameter of target lesions, taking as reference the baseline sum longest diameter. In some embodiments, the methods of the present invention may result in stable disease of target lesions, comprising neither sufficient shrinkage to qualify for partial response nor sufficient increase to qualify for progressive disease, taking as reference the smallest sum longest diameter since the treatment started. In such an embodiment, progressive disease is characterized by at least a 20% increase in the sum of the longest diameter of target lesions, taking as reference the smallest sum longest diameter recorded since the treatment started or the appearance of one or more new lesions. The sum must demonstrate an absolute increase of 5 mm.

In another embodiment, the methods of the present invention result in the treatment of non-target lesions. In this embodiment, the methods of the present invention may result in a complete response, comprising the disappearance of all nontarget lesions. In some embodiments, the methods of the present invention result in persistence of one or more nontarget lesion(s), while not resulting in a complete response or progressive disease. In such an embodiment, progressive disease is characterized by unequivocal progression of existing nontarget lesions, and/or the appearance of one or more new lesions.

In some embodiments, the methods of the present invention result in a beneficial overall response rate, such as an overall response rate according to RECIST v.1.1. In those embodiments, the methods of the present invention result in an overall response that is a complete response wherein the subject exhibits a complete response of target lesions, a complete response of nontarget lesions, and no new lesions. In other embodiments, the methods of the present invention result in an overall response that is a partial response, wherein the subject exhibits a complete response for target lesions, non-complete response and non-progressive disease for non-target lesions, and no new lesions. In other embodiments, the methods of the present invention result in an overall response that is a partial response, wherein the subject exhibits a partial response for target lesions, non-progressive disease for non-target lesions, and no new lesions. In another embodiment, the methods of the present invention result in an overall response that is stable disease wherein the subject exhibits stable disease of target lesions, non-progressive response for non-target lesions, and no new lesions.

In some embodiments, the methods of the present invention result in an increased duration of overall response. In some embodiments, the duration of overall response is measured from the time measurement criteria are met for complete response or partial response (whichever is first recorded) until the first date that recurrent or progressive disease is objectively documented (taking as reference for progressive disease the smallest measurements recorded since the treatment started). The duration of overall complete response may be measured from the time measurement criteria are first met for complete response until the first date that progressive disease is objectively documented. In some embodiments, the duration of stable disease is measured from the start of the treatment until the criteria for progression are met, taking as reference the smallest measurements recorded since the treatment started, including the baseline measurements.

In yet other embodiments, the methods of the present invention result in improved overall survival rates. For example, overall survival may be calculated from the date of enrollment to the time of death. Patients who are still alive prior to the data cutoff for final efficacy analysis, or who dropout prior to study end, will be censored at the day they were last known to be alive.

In other embodiments, the methods of the prevent invention result in progression-free survival. For instance, progression-free survival may be calculated from the date of documenting relapse (or other unambiguous indicator of disease development), or date of death, whichever occurs first. Patients who have no documented relapse and are still alive prior to the data cutoff for final efficacy analysis, or who drop out prior to study end, will be censored at the date of the last radiological evidence documenting absence of relapse.

In some embodiments, the methods of the present invention result in a beneficial overall response rate, such as an overall response rate according to mRECIST. In those embodiments, the methods of the present invention result in an overall response that is a complete response wherein the subject exhibits a complete response of target lesions, a complete response of nontarget lesions, and no new lesions. In other embodiments, the methods of the present invention result in an overall response that is a partial response, wherein the subject exhibits a complete response for target lesions, non-complete response and incomplete response for non-target lesions, and no new lesions. In other embodiments, the methods of the present invention result in an overall response that is a partial response, wherein the subject exhibits a partial response for target lesions, non-progressive disease for non-target lesions, and no new lesions. In another embodiment, the methods of the present invention result in an overall response that is stable disease wherein the subject exhibits stable disease of target lesions, non-progressive response for non-target lesions, and no new lesions.

In some embodiments, the methods of the present invention result in a beneficial overall response rate, such as an overall response rate according to iRECIST.

According to another embodiment, the methods of the present invention include a method for treating a liver metastasis of uveal melanoma, wherein the administration of SD-101 results in a reduction of tumor burden. In some embodiments, the tumor burden is reduced by about 10%, by about 20%, by about 30%, by about 40%, by about 50%, by about 60%, by about 70%, by about 80%, by about 90%, or by about 100%.

According to another embodiment, the methods of the present invention include a method for treating a liver metastasis of uveal melanoma, wherein the administration of SD-101 results in a reduction of tumor progression. In some embodiments, tumor progression is reduced by about 10%, by about 20%, by about 30%, by about 40%, by about 50%, by about 60%, by about 70%, by about 80%, by about 90%, or by about 100%.

According to another embodiment, the methods of the present invention include a method for treating a liver metastasis of uveal melanoma, wherein the administration of SD-101 reprograms the liver MDSC compartment to enable immune control of liver metastases and/or improves responsiveness to systemic anti-PD-1 therapy through elimination of MDSC. In some embodiments, the methods of the present invention are superior in controlling MDSC. In some embodiments, the methods of the present invention include a method for treating a liver metastasis of uveal melanoma, wherein the administration of SD-101 reduces the frequency of MDSC cells (CD11b+Gr1+), monocytic MDSC (M-MDSC; CD11b+Ly6C+) cells, or granulocytic MDSC (G-MDSC; CD11b+LY6G+) cells. According to another embodiment, the methods of the present invention enhance M1 macrophages. According to yet another embodiment, the methods of the present invention decrease M2 macrophages.

In another embodiment, the methods of the present invention increase NFκB phosphorylation. In yet an additional embodiment, the methods of present invention increase IL-6. In another embodiment, the methods of the present invention increase IL10. In yet an additional embodiment, the methods of present invention increase IL-29. In another embodiment, the methods of the present invention increase IFNα. As a further embodiment, the methods of the present invention decrease STAT3 phosphorylation.

The present invention will be further illustrated and/or demonstrated in the following Example, which is given for illustration/demonstration purposes only and is not intended to limit the invention in anyway.

### Example 1

In the present example, it was hypothesized that regional intra-vascular infusion of an exemplary TRL9 agonist would enhance the responsiveness to a systemically infused CPI.

In this regard, mice with established MC38-CEA-Luc LM were treated with a regional delivery of a TLR9 agonist, i.e. ODN-2395 (30 µg/mouse), with or without an intraperitoneal delivery of the CPI, e.g. anti-PD-1 antibody (250 µg/mouse). Eight to twelve week male C57/BL6 mice were challenged intra-splenic with 0.5e6 MC38-CEA-Luc cells for a week. Bioluminescence was measured by IVIS and mice were randomized on D0 prior to treatment with 30 µg/mouse ODN2395 via PV with/without 250 µg/mouse of anti-PD1 antibody via IP on D0, D+3 and D+6. PBS treated mice via PV was used as control. Tumor progression was monitored on D+2, D+4, D+7, D+10 and D+12.

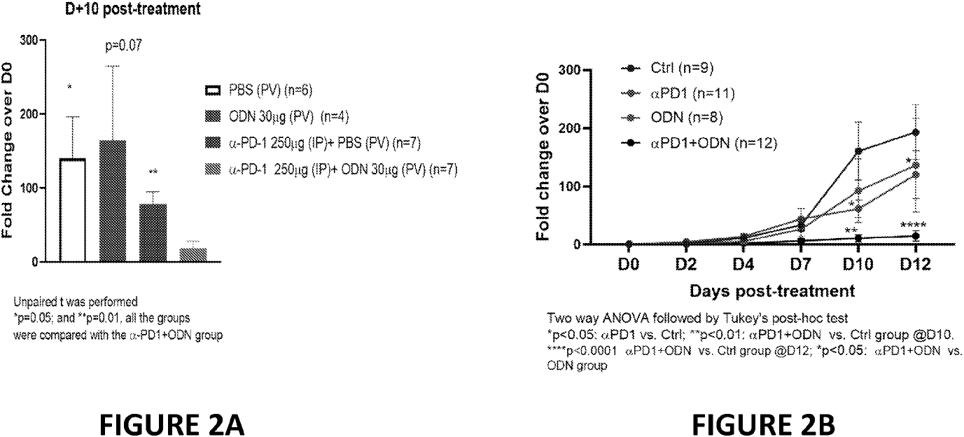

FIGS. 2A-2B illustrates the effect of a combination of an exemplary TLR9 agonist and the CPI on tumor progression. As depicted in the figure, control of LM growth was significantly higher with combinatorial treatment as compared to the anti-PD-1 (p<0.01) or control (e.g. PBS) treatments (p<0.05). In FIG. 2A, PBS (PV), ODN 30 µg (PV), anti-PD-1 250 µg (IP) + PBS (PV), and anti-PD-1 250 µg (IP) + ODN 30 µg (PV) are shown in the graph (left-to-right, PBS closest to the Y-axis). In FIG. 2B, tumor growth was monitored by IVIS imaging on D+2, D+4, D+7, D+10 and D+12. Tumor progression was analyzed by 2-Way ANOVA followed by Tukey’s post-hoc test. In the graph, fold change over D0 is exhibited for Ctrl, anti-PD-1, ODN, and anti-PD-1 + ODN over time, with Ctrl showing the greatest fold change over D0 and anti-PD-1 + ODN showing the least.

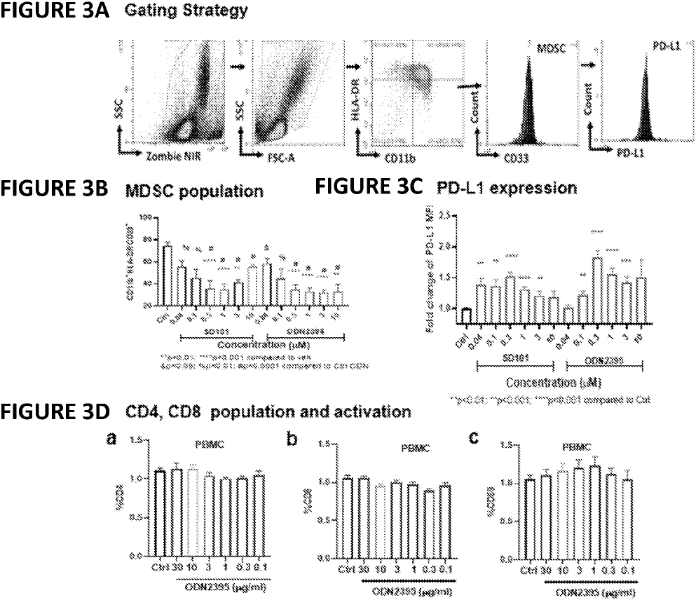

To study the impact of TLR9 activation on human MDSC (hu-MDSC), healthy donor peripheral blood mononuclear cells (PBMCs) were treated with ODN-2395 or SD-101. It was found that both reduced the hu-MDSC (CD11b+CD33+HLADR-) population in a nonlinear dose-dependent manner with an increase in PD-L1 expression as determined by flow cytometry (FC) analysis, as depicted in FIGS. 3A-3D. In this study, human PBMCs were isolated from the Leukoreduction Reservoir System (LRS) chambers. 1e6/ml PBMCs were treated with increasing concentrations (0.04-10 µM) SD-101, ODN2395 and ctrl ODN5328 (1 µM) for 48 hours. In FIG. 3A, the gating strategy for phenotypic analysis of MDSC and PD-L1 expression is illustrated. In FIGS. 3B-3C, MDSC population and their corresponding PD-L1 expression were evaluated. Four donors with three replicates were used, and data represented as mean ± SEM. In FIG. 3D, (a) CD4, (b), CD8 (c) and CD69 were evaluated by FC. Three donors with three replicates were used. These figures demonstrate that TLR9 Stimulation with ODN2395 or SD-101 inhibits MDSC generation from PBMCs.

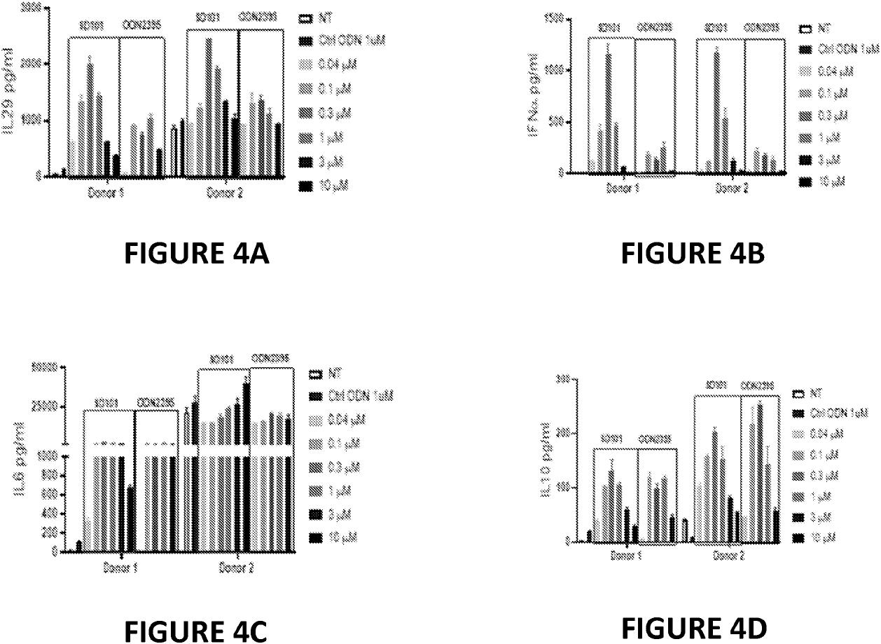

Moreover, by using Luminex, it was demonstrated that ODN-2395 and SD-101 enhanced expression of IL-29, IFNα, and NFκB, along with downstream cytokines IL-6 and IL-10. In FIGS. 4A-4D, human PBMC were treated with increasing doses of (0.04-10 µM) SD-101 (left box), (0.04-3 µM) ODN2395 (right box) and ctrl ODN5328 (1 µM) for 48 hours. Cell supernatants were analyzed for IL-29 (FIG. 4A), IFNα (FIG. 4B), IL-6 (FIG. 4C), and IL-10 (FIG. 4D) using Luminex analysis. Two donors and two replicates were used. NT, Ctrl ODN, and increasing doses (0.04-3 µM) are shown starting left-to-right on the X-axis of each group. FIGS. 3A-3D show that TLR9 stimulation with ODN2395 or SD-101 enhances NFκB and IFNα regulated cytokine production.

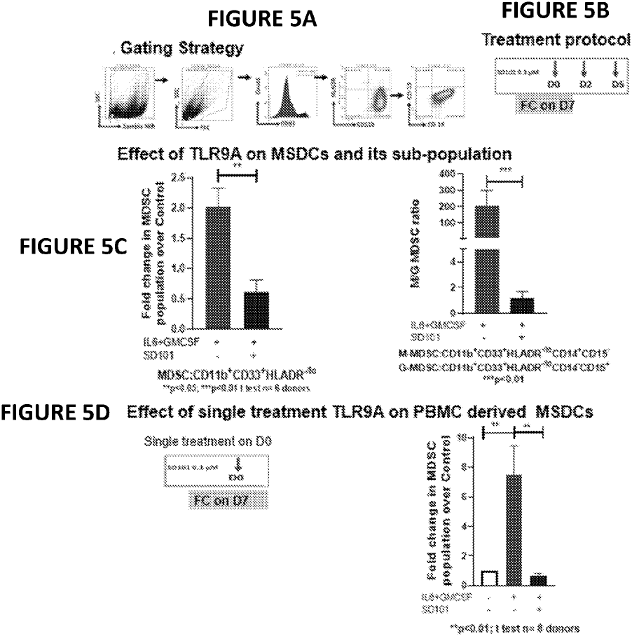

Further, to investigate the effect of SD-101 in modulating the differentiation of hu-MDSC from human PBMC, the human PBMC was treated with IL6+GM-CSF in the presence or absence of SD-101, as shown in FIGS. 5A-5D. In this study, human PBMC were treated with 20 ng/ml of IL6 and GM-CSF for 7 days with/without 0.3 µM SD-101 either intermittently or once for 48 hrs. In FIG. 5A, a gating strategy for phenotypic analysis of MDSC (CD11b+CD33+HLADR-) is illustrated. In FIG. 5B, the treatment protocol is illustrated. In FIG. 5C, the effect of TLR9A on MDSCs and its sub-population is illustrated. In FIG. 5D, PBMCs were differentiated into MDSC in the presence of IL6 and GM-CSF and treated with/without 0.3 µM SD-101 once for 48 hrs and after 7 days, FC was performed to monitor MDSC population (eight donors). Data represented as mean ± SEM. In this regard, by using FC analysis, it was found that SD-101 blocked hu-MDSC development induced by IL6+GM-CSF, preferentially limited the more immunosuppressive monocytic MDSC subtype, and also drove M1 macrophage polarization. Further, treatment of SD-101 only once for 48 hours was sufficient to inhibit hu-MDSC differentiation for two weeks. Thus, it was shown that TLR9 stimulation with SD-101 inhibits human MDSC programming.

In summary, TLR9 stimulation enhances the ability of checkpoint therapy to control liver metastases in a murine model. TLR9 agonists inhibit PBMC-derived MDSCs and enhance MDSC PD-L1 expression without affecting T cell populations. ODN2395 and SD-101 modulate the production of IFNα, IL29 (IFNα dependent cytokines), IL6 and IL10 (NFκB dependent cytokines) in a biphasic manner. SD-101 inhibits MDSC programming and single treatment is sufficient to elicit this inhibitory effect.

Thus, both the in-vitro and in-vivo findings suggest that regional TLR9 stimulation in a model of LM improves responsiveness to systemic anti-PD-1 therapy through elimination of MDSC, with the effect on blood hu-MDSC being confirmed in-vitro. Further, increased PD-L1 expression in response the TLR9 stimulation among MDSC further enhanced the anti-PD-1 effect. Therefore, combining regional infusions of a TLR9 agonist with systemic anti-PD-1 agents improves responsiveness to anti-PD-1 therapy by suppressing MDSC programming may be used to treat liver tumors and to provide intrahepatic immunosuppression.

### Example 2

In the present example, it was hypothesized that regional intra-vascular infusion of TRL9 agonists would reprogram the liver MDSC compartment to enable immune control of liver metastases (LM). The effect of a class C TLR9 agonist, e.g. ODN-2395, was evaluated in inhibiting LM progression and its impact on the liver MDSC population. In particular, a regional intra-vascular infusion of ODN-2395 was compared to systemic delivery of ODN-2395 with respect to control of LM progression and impact on the liver MDSC population size.

C57/BL6 mice were challenged with 2.5e6 MC38-CEA-Luc cells via intra-splenic route. After a week, the mice were treated with 1, 3, 10, or 30 µg of ODN-2395 via portal vein (PV: regional), or 30 µg intravenously (systemic) through the tail vein (TV). Administration of an agent through the murine portal vein results in regional administration to the animal liver. The tumor burden was measured at 24 and 48 hours post-ODN administration by evaluating bioluminescence. CD45+ cells were isolated, and FACS analysis was performed to quantify MDSCs, monocytic MDSCs (M-MDSC) and M1-macrophage subsets, along with signaling events downstream of TLR9.

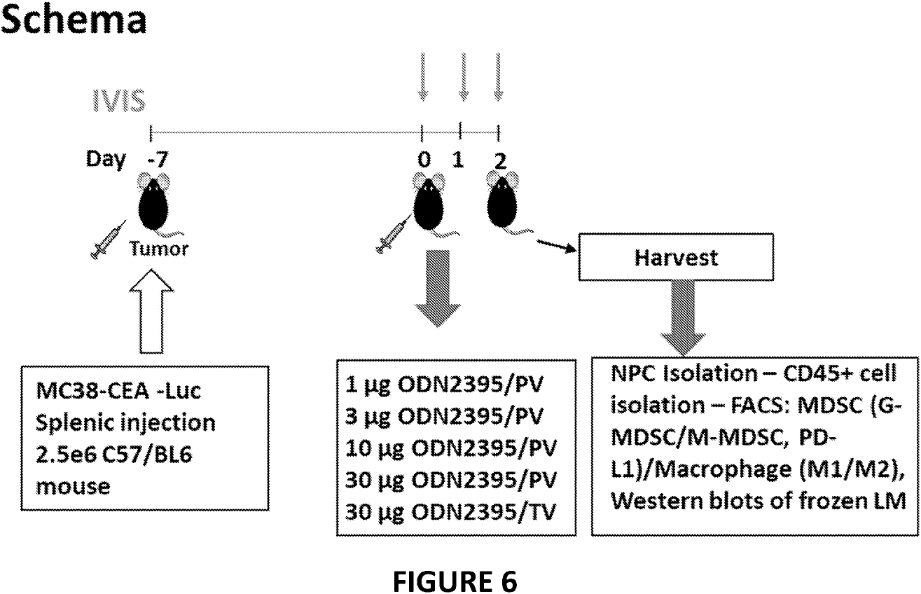

FIG. 6 illustrates the schema/method for developing LM and the treatment protocol. Eight to twelve week old male C57/BL6 mice were challenged via the intra-splenic route with 2.5e6 MC38-CEA-Luc cells, which were allowed to grow for one week. Bioluminescence values were determined by IVIS, and mice were randomized accordingly and treated with 1, 3, 10, 30 µg/mouse ODN-2395 via PV and 30 µg/mouse ODN-2395 via TV. For the subsequent study on D+2 post-treatment, mice were sacrificed, and the livers were harvested to isolate CD45⁺ cells. Isolated CD45⁺ NPCs were then evaluated for MDSCs and macrophages.

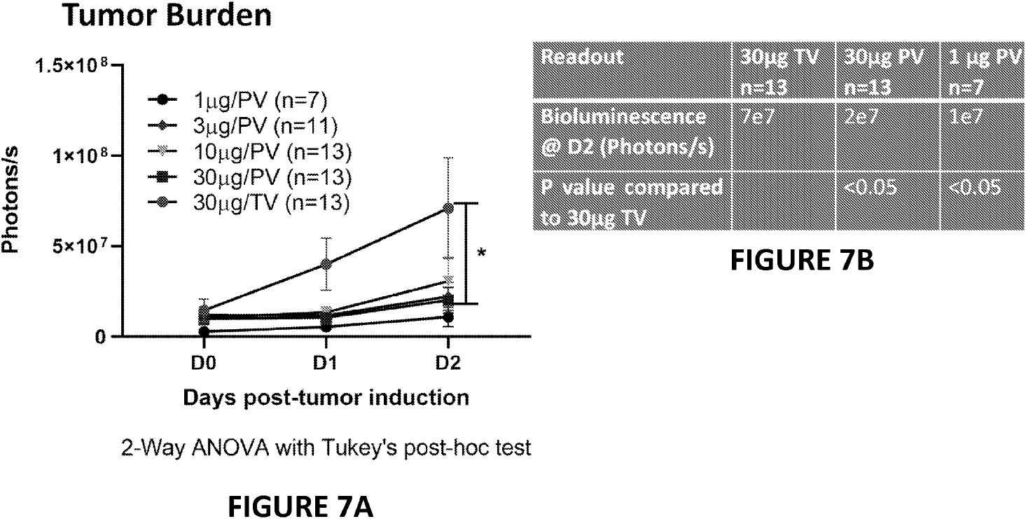

FIGS. 7A-7B illustrate the effect of ODN-2395 on tumor progression. In particular, FIG. 7A depicts the tumor growth/burden at the day of treatment (D0), D1, and D2 for 1, 3, 10, 30 µg/mouse ODN-2395 via PV and 30 µg/mouse ODN-2395 via TV. The tumor progression was analyzed by 2-Way ANOVA followed by Tukey’s post-hoc test (*p<0.05). Further, FIG. 7B depicts the bioluminescence and P value for tumor burdens that were treated by 30 µg ODN2395 via PV and TV. As depicted in FIGS. 7A-7B, 30 µg of ODN-2395 administered via PV (as compared to TV) reduced the tumor burden significantly (p<0.01).

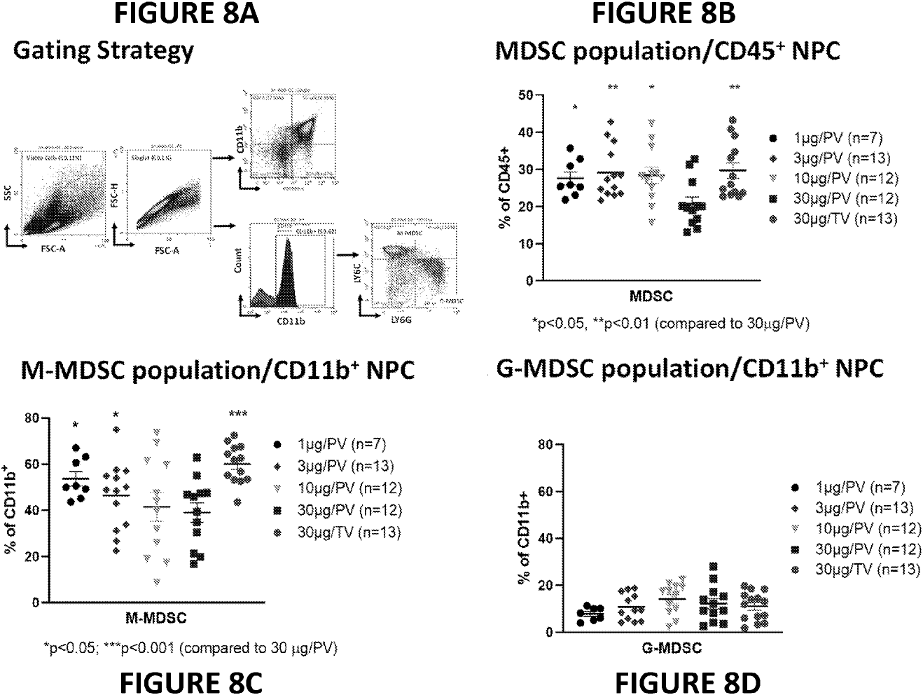

FIGS. 8A-8D illustrate the effect of ODN-2395 on the MDSC population in LM. In this regard, FIG. 8A illustrates a gating strategy to analyze CD45⁺ cells isolated from the LM by FACS. Further, FIGS. 8B, 8C, and 8D illustrate the measured MDSC cells (CD11b+Gr1+), monocytic MDSC (M-MDSC; CD11b+Ly6C+) cells, and granulocytic MDSC (G-MDSC; CD11b+LY6G+) cells, respectively, for 1, 3, 10, 30 µg/mouse ODN-2395 via PV and 30 µg/mouse ODN-2395 via TV. As depicted in FIGS. 8B and 8C, 30 µg of ODN-2395 via PV was superior in controlling MDSC and M-MDSC as compared to the mice that received 30 µg of ODN-2395 via TV, suggesting increased tumor-suppressive TME that can be favorably reprogrammed by ODN-2395, particularly when delivered at 30 µg ODN-2395 via PV.

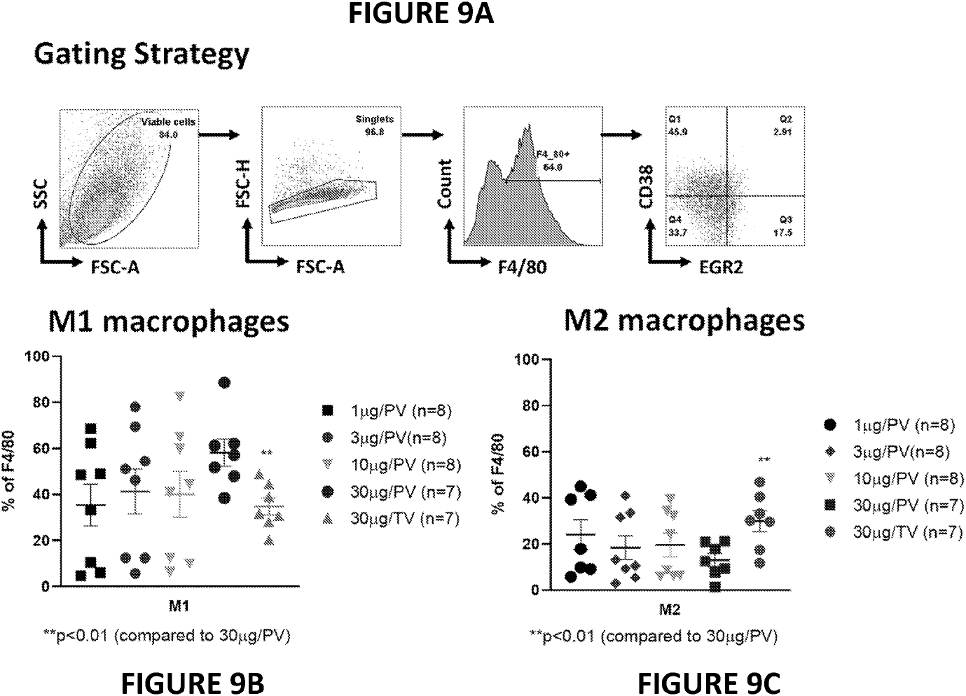

FIGS. 9A-9C illustrate the effect of ODN-2395 on the M1- and M2-macrophage populations in LM. In this regard, FIG. 9A illustrates a gating strategy for analyzing the CD45⁺ cells isolated from the LM for M1- and M2-macrophages. In particular, a phenotypic analysis is shown. FIGS. 9B and 9C illustrate the measured M1-macrophage cell population (F4/80⁺CD38⁺EGR2⁻) and M2-macrophage cell population (F4/80⁺CD38⁻EGR2⁺) for 1, 3, 10, 30 µg/mouse ODN-2395 via PV and 30 µg/mouse ODN-2395 via TV. In this regard, mice that received ODN-2395 via PV had significantly increased M1 macrophage populations and reduced M2 populations compared to and 30 µg via TV. This data suggests ODN-2395, particularly 30 µg via PV, polarized CD11b+F4/80+ monocytic cells towards a pro-inflammatory/anti-tumorigenic M1 macrophage and reduced pro-angiogenic M2 population, in addition to MDSC.

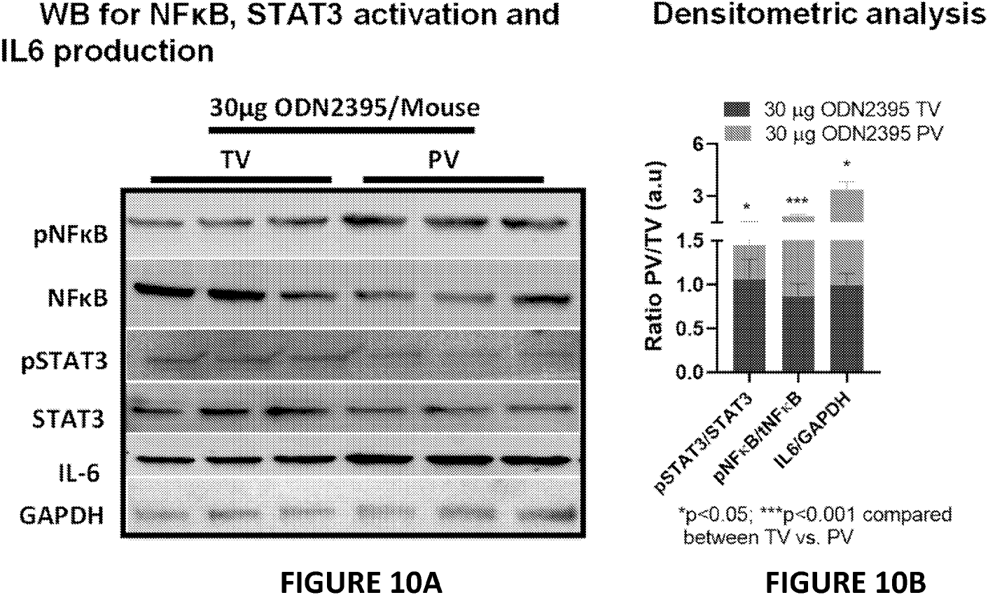

FIGS. 10A-10B illustrate the effect of ODN-2395 on NFκB signaling. In particular, FIG. 10A illustrates Western blotting for pNFκB (p65^{S536}), total NFκB, pSTAT3^{Y705}, STAT3, and IL-6 for 30 µg ODN2395 by PV and TV, with GAPDH used as a protein control. Further, FIG. 10B depicts the densitometric analysis for 30 µg ODN-2395 by PV and TV. In this regard, 30 µg of ODN-2395, when infused via PV, increased NFκB phosphorylation with a concomitant increase in IL-6 and decreased STAT3 phosphorylation in the LM compared to TV injection.

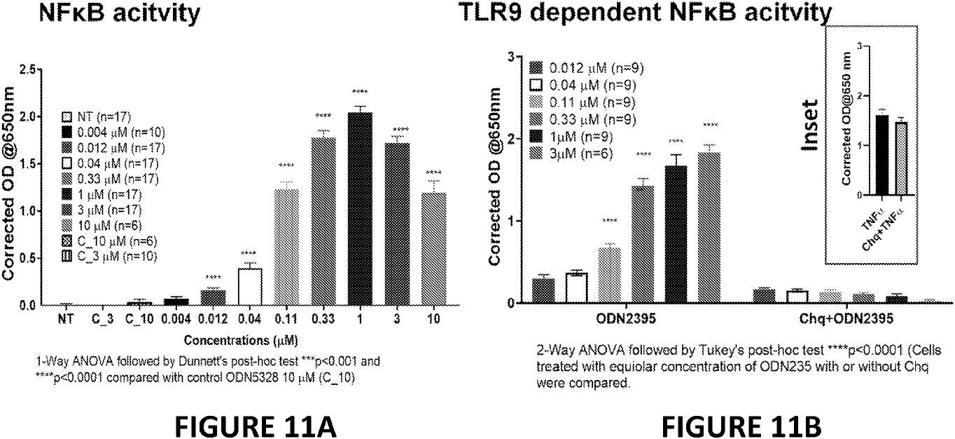

FIGS. 11A-11B illustrate effect of ODN-2395 concentration on NFκB signal activity, where, in a reporter-based assay, HEK293-Blue cells were treated with ODN-2395 and SD-101 at increasing doses (0.004-10 µM) for 21 hours. This was done in order to evaluate dose-dependent effects of ODN-2395 in activating NFκB signaling via TLR9. In this regard, the release secreted embryonic alkaline phosphatase (SEAP) in FIG. 11A was determined by measuring the absorbance at 650 nm. Further, FIG. 11B depicts the effect of chloroquine (Chq) on the NFκB signal activity. In this regard, cells were pre-treated with chloroquine (1 µg/ml) for 45 minutes before the addition of ODN-2395 at increasing concentrations (0.012-3 µM) for 21 hours and absorbance at 650 nm was measured. The absorbance at 650 nm of cells treated with TNFα with/without Chq pretreatment is depicted in FIG. 11B. As depicted in FIG. 11B, chloroquine inhibited the TLR9 agonist mediated activation of NFκB, demonstrating that ODN-2395 activates NFκB pathway by interacting with TLR9.

In view of the above, it was determined that in vivo PV delivery of 30 µg of ODN-2395 decreased tumor burden; decreased the frequency of MDSCs (predominantly the more immunosuppressive M-MDSCs subpopulation); and enhanced pro-inflammatory/ anti-tumorigenic M1 macrophages, with a concomitant decrease in immunosuppressive M2 macrophages. Further, at the molecular level, ODN-2395 increased the phosphorylation of NFκB along with IL6 expression and decreased phosphorylation of STAT3. In addition, the in vitro SEAP assay confirmed that ODN-2395 mediated NFκB activation is TLR9 dependent.

In summary, 30 µg of ODN-2395 administered via PV was more efficient (as compared to TV) in reducing tumor burden at 24 hours and persisted up to 48 hours. Regional delivery of ODN-2395 also reduced the frequency of MDSCs, predominantly the more immunosuppressive M-MDSCs subpopulation in LM. Further, regional delivery of ODN-2395 also enhanced pro-inflammatory/anti-tumorigenic M1 macrophages. In addition, using an NFκB-dependent soluble alkaline phosphatase assay (e.g. SEAP), it was determined that ODN-2395 dose-dependently enhanced NFκB transcription factor activity (p<0.001). Further, Western blot data of tumor lysates illustrated that ODN delivery by PV significantly increased NFκB (pP65) activity and production of IL-6 as well as reduced STAT3 activity relative to IV.

### Example 3

In this example, an SD-101 HAI/PEDD general toxicology study was performed on domestic pigs. This study was conducted using the TriNav® catheter for direct hepatic arterial administration at dosages of 0 (vehicle) using a volume of 10 mL/kg/day. Dosages to be evaluated were 0 (vehicle), 2, 4, and 8 mg SD-101. Three SD-101 administrations were given to 2 pigs /group on Days 0, 7, and 14 with the necropsy occurring on Day 15. Standard assessments were performed throughout the study, including clinical observations, body weight and food consumption. Complete veterinary physical examinations were conducted prior to the drug administration period and after the second and third administrations of SD-101.

Serial blood samples for toxicokinetic (TK) analysis (and up to 2 metabolites) were collected on Days 0 and 14 for calculations of SD-101 exposure and disposition. Samples were also taken on these same days for possible anti-drug antibody (ADA) analyses. Serum samples were collected for cytokine analyses pre-treatment and on Days 0 (prior to administration) and 14. A thorough hematology, clinical chemistry and coagulation panel was conducted pre-treatment and just prior to necropsy. The following organ weights were determined, and microscopic histopathology exams were conducted in: the liver (4 lobes), lung, spleen, thymus, kidneys, heart, lymph nodes (draining and non-draining), gut associated lymphoid tissue, bone marrow, brain and gross lesions. Tissue levels of SD-101 and up to 2 metabolites were determined in the liver (4 lobes), lung, spleen, thymus, kidneys, heart, lymph nodes (draining and non-draining), gut associated lymphoid tissue, bone marrow, brain, and gross lesions. Across all pigs in the study, there was no evidence of hepatotoxicity following infusion of SD-101 into the hepatic artery.

Further to the above, a repeat study was conducted in which animals received a single administration of SD-101 at doses of 0, 0.5, 4, or 8 mg. Consistent with the prior study, no treatment-related adverse effects were observed. Importantly, serum pharmacokinetic analyses demonstrated that systemic levels of SD-101 following HAI in swine were 100-fold lower than those observed in primate studies following subcutaneous injection.

### Example 4

In this example, a comparison between the administration a TLR9 agonist via PEDD/HAI, e.g. with the TriNav® Infusion System, and the administration of TLR9 agonist via needle injection was studied. In particular, the study focused on the route of administration (direct injection into the hepatic tissue via needle injection versus local-regional PEDD infusion) on the intrahepatic localization of fluorescent-tagged Toll-like receptor 9 (TLR9) Class C agonists SD-101 (Cy5.5-SD-101) and tool molecule oligo deoxynucleotide (ODN) 2395 Oligo (IRD800-ODN 2395).

To evaluate the impact of a PEDD device on therapeutic distribution, a porcine model was selected based on similarities in liver vasculature, cellular structure, and internal organ structure relative to human liver. Porcine models have been used extensively in investigations of local delivery of therapeutics. The liver vascular anatomy is compatible with the size range indication for the PEDD TriNav® device (1.5-3.5 mm diameter).

The study was conducted on 8-11 week old female Yorkshire Cross swine ranging in weight from 45-65 kg.

The TLR9 agonist SD-101 sequence oligo was synthesized and conjugated to the Cy5.5 (ex. 685 nm, em. 706 nm) fluorophore.

The TLR9 agonist ODN 2395 sequence oligo was synthesized and conjugated to the IRDye800 (ex. 791 nm, em. 809 nm) fluorophore.

### Study Design

A total of 8 healthy female swine were selected for the study. The first cohort animals (n=4) received IRD800-ODN 2395 via HAI with a PEDD device (TriNav®, Cat No TNV-21120-35) followed by needle injection of Cy5.5-SD-101. The second cohort animals (n=4) received Cy5.5-SD-101 via HAI with a PEDD device followed by needle injection of IRD800-ODN 2395.

### Needle Injection

A 21 gauge, 15 cm long percutaneous access needle was inserted into the right lateral or right medial lobe of the liver under ultrasound guidance. The 3 mL volume of oligo solution was then injected at a rate of 0.3-0.6 mL/sec by hand. This rate and volume are consistent with clinical practice using needle injection of therapeutics into tumors.

### HAI

HAI was conducted using the TriNav® Infusion System. The TriNav®is a single lumen catheter equipped with a one-way valve that responds dynamically to local pressure changes, such as those arising from the cardiac cycle or generated by infusion. The valve structure modulates distal vascular pressures and blood flow. This in turn may alter therapeutic distribution and first-pass absorption due to increased contact time within the vasculature.

The Seldinger technique was used to gain access through the femoral artery. A 5F introducer sheath was secured at the site. A 5F angiographic catheter was used to perform angiography to identify hepatic arterial anatomy. A 1.5- to 3.5-mm diameter vessel (mean 2.59 mm±0.21 mm SE) feeding the left medial and/or left lateral lobe was then selected. The TriNav® Infusion System was tracked into the target vessel location. An invasive blood pressure (IBP) transducer connected to a Quantien pressure Monitor was used to measure pressure through the lumen of the infusion system (distal to the microvalve) and through the lumen of the angiographic catheter (proximal to the microvalve). The 10 mL therapeutic syringe was then placed into a syringe pump and the solution was infused through the TriNav® device at a rate of 2 ml/min for a total duration of 5 minutes.

### Blood Sample Collection

Procedure and therapeutic impact on liver enzymes (alanine aminotransferase [ALT] and aspartate aminotransferase [AST]) and were assessed. Blood was drawn prior to intervention, post device placement within the target vessel, immediately after HAI (t=0) and for every 10 minutes thereafter for a total of 90 minutes.

### Tissue Preparation

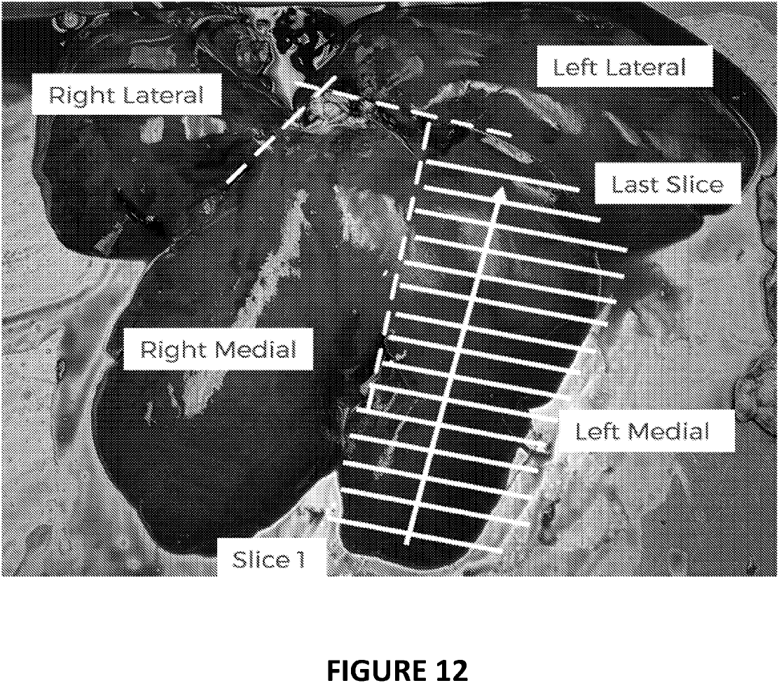

Animals were sacrificed after infusion and blood sample collection and the liver was removed. Each lobe of the liver was separated and near-IR imaging with a Pearl Trilogy Imaging System was performed to identify patterns of drug uptake. Each lobe was then cut into 1 cm thick sections (FIG. 12) and separately imaged (85 µm resolution, 700 nm, 800 nm, and white light channels) on both tissue faces using the Pearl system. The full liver volume was analyzed for the presence of the ODN tool compound within the tissue.

### Therapeutic Signal Quantification

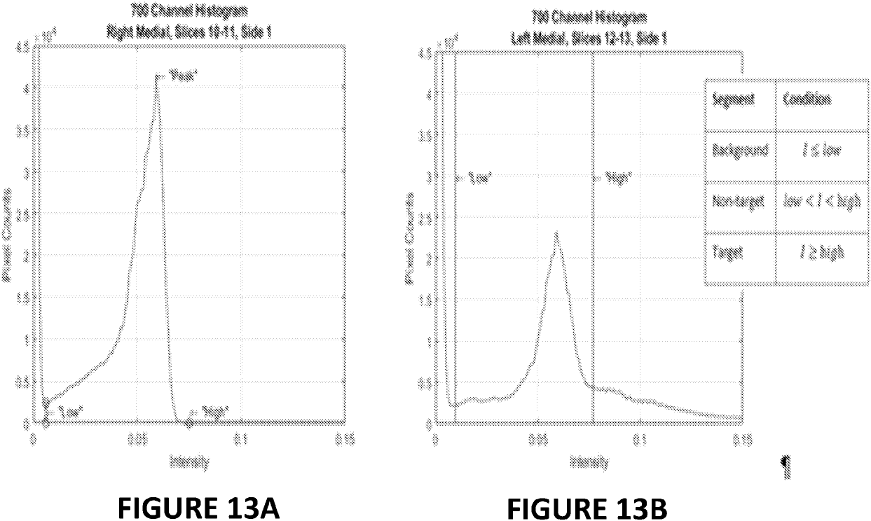

Images of tissue produced by the Pearl Imaging system consisted of separate 700 nm and 800 nm fluorescence channels. Within the image, distinct emission intensity levels were observed for the background sample preparation plate, normal untreated liver tissue, and treated liver tissue. A custom designed MATLAB graphical user interface (GUI) was developed to identify these discrete regions of signal (FIGS. 13A and 13B). Slices (n=3) taken from untreated regions of the liver of each animal were imaged to produce a histogram of pixel luminous intensity values. A “low” intensity (I) value limit was identified for pixel intensities associated with the sample preparation plate. A “high” intensity value limit was determined at the maximum pixel intensity measured in untreated non-target tissue. An average was taken for each of the Low (n=3) and High (n=3) measurements to determine tissue threshold values. Tissue slices were then processed based on the threshold values.

Signal arising from treated target tissue, identified by a pixel intensity greater than that of the “High” threshold, was established for the 700 nm (Cy5.5-SD-101) and 800 nm (IRD800-ODN 2395) tool compounds. The total signal intensity in luminous units (lu) was calculated by the summation of the lu for pixels meeting these criteria. This produced data corelated to the signal observed for the needle injection (n=4 for IRD800-ODN2395, n=4 for Cy5.5-SD-101, n=8 total measurements) and the HA infusion (n=4 for IRD800-ODN2395, n=4 for Cy5.5-SD-101, n=8 total measurements). The mean lu for the two sides of each slice was determined. The summation of these measurements for all slices was then calculated for each subject and the lu totals for the two infusion methods were then compared using a two-sample equivalence test at a 95% confidence interval.

Volume of coverage was calculated by taking the mean of the number of pixels displaying intensity greater than the “High” threshold for the two sides of each slice. This value was then converted into a tissue volume (85 µm x 85 µm pixel x 1 cm slice depth) for all tissue slices (needle injection (n=4 for IRD800- ODN2395, n=4 for Cy5.5-SD-101, n=8 total measurements) and the HAI (n=4 for IRD800-ODN2395, n=4 for Cy5.5-SD-101, n=8 total measurements)). Tissue volume for the two infusion methods were then compared using a two-sample equivalence test at a 95% confidence interval.

### Results

### Tissue Imaging

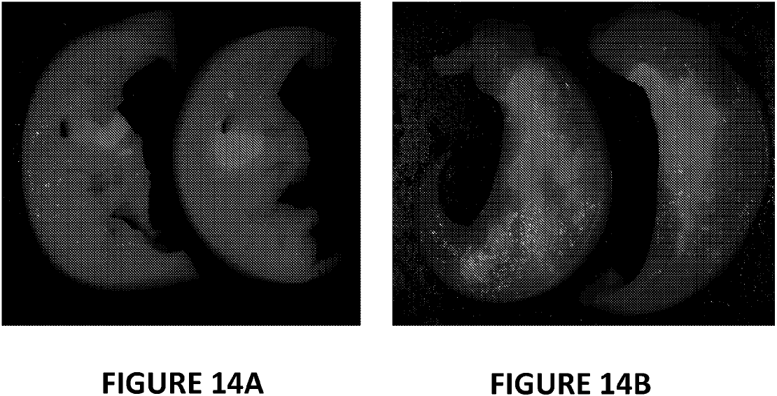

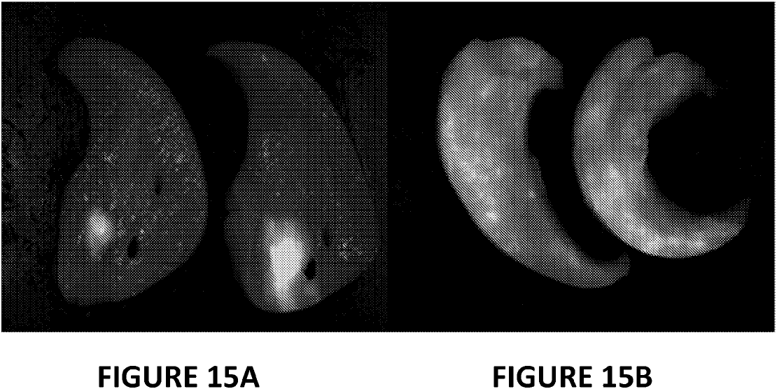

Near-IR imaging of liver tissue displayed distinct differences in the pattern of labelled ODN distribution when therapy was delivered in the local arterial network using a PEDD device (FIG. 14B and FIG. 15B) relative to needle injection (FIG. 14A and FIG. 15A).

### Therapeutic Delivery

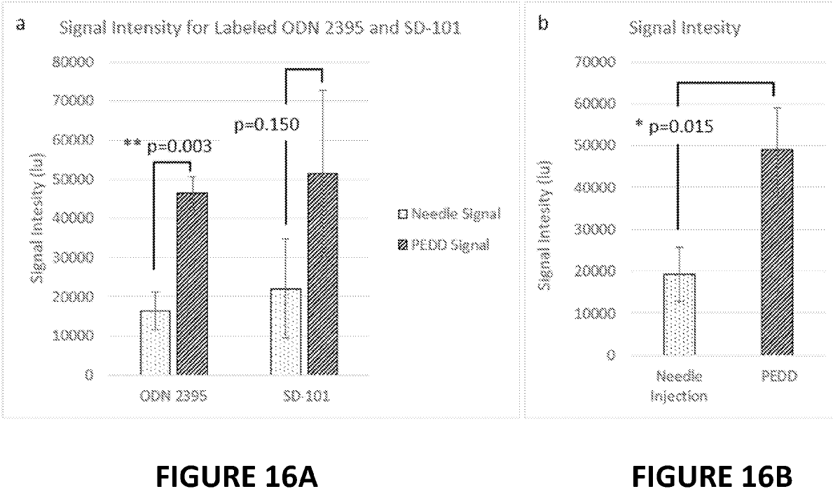

Analysis of the total signal recorded in hepatic tissues was performed for each of the ODNs. The signal intensity measured for labeled ODN 2395 was significantly greater when infused using a PEDD device (n= 4, 46483±4285lu SE) than with injection by needle (n=4, 16438±4793lu SE)(p=0.003) (FIG. 16A). Signal intensity for labeled SD-101 (n=4, 51511±21267lu SE) was not significantly different than needle injection (n=4, 22112±12648lu SE)(p=0.15) due to higher variability between animals (FIG. 16A). The combined signal intensity of both ODNs delivered by PEDD (n=8, 48997±10088 lu SE) was significantly greater than when delivered by needle injection (n=8, 19275±6352 lu SE)(p=0.015) (FIG. 16B).

### Therapeutic Coverage

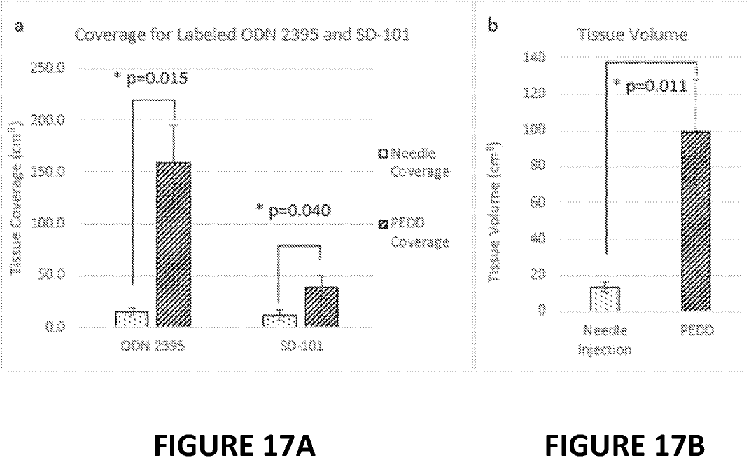

Analysis of the total signal recorded in hepatic tissues was performed for each of the ODNs. The tissue coverage measured for labeled ODN 2395 was significantly greater when infused using a PEDD device (n= 4, 159.2:J::36.6 cm³ SE) than with injection by needle (n=4, 15.7±3.3 cm³ SE) (p=0.015) (FIG. 17A). Tissue coverage for labeled SD-101 (n=4, 38.8±10.9 cm³ SE) was also significantly greater than needle injection (n=4, 11.3±4.7cm³ SE) (p=0.04) (FIG. 17A). The combined signal intensity of both compounds delivered by PEDD (n=8, 99.0±28.8 cm³ SE) was significantly greater than when delivered by needle injection (n=8, 13.5±2.9 cm³ SE)(p=0.011) (FIG. 17B).

### Liver Enzyme Levels

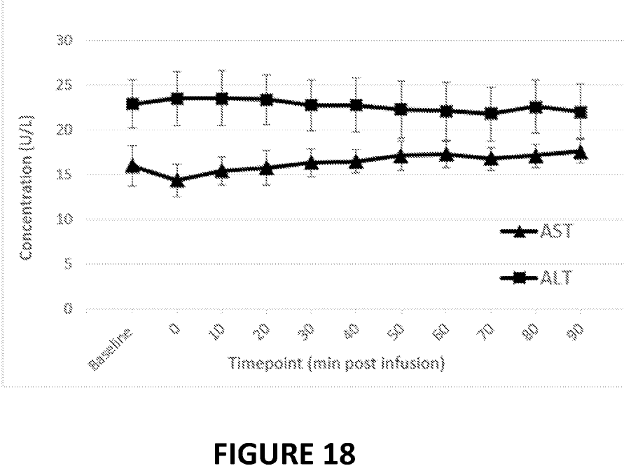

Using Minitab Software (Minitab LLC, Chicago, IL) paired t-tests were conducted to determine if AST or ALT levels experienced a significant change relative to pre-procedure baseline levels. No significant changes were observed for either AST or ALT over the course of blood collection (Table 2 and FIG. 18).

TABLE 2

| Postdose Timepoint (min) | Mean ALT (SEM) (U/L) | p-Value ^{a} | Mean AST (SEM) (U/L) | p-Value ^{a} |
|---|---|---|---|---|
| Baseline | 23 (2.7) | | 16 (2.2) | |
| 0 | 24 (3.0) | 0.879 | 14 (1.8) | 0.580 |
| 10 | 24 (3.1) | 0.881 | 15 (1.6) | 0.823 |
| 20 | 23 (2.8) | 0.899 | 16 (1.9) | 0.934 |
| 30 | 23 (2.9) | 0.975 | 16 (1.6) | 0.893 |
| 40 | 23 (3.0) | 0.976 | 17 (1.2) | 0.848 |
| 50 | 22 (3.2) | 0.883 | 17 (1.6) | 0.690 |
| 60 | 22 (3.3) | 0.862 | 17 (1.5) | 0.648 |
| 70 | 22 (3.0) | 0.785 | 17 (1.3) | 0.776 |
| 80 | 23 (3.0) | 0.951 | 17 (1.3) | 0.672 |
| 90 | 22 (3.1) | 0.834 | 18 (1.4) | 0.550 |

ALT = alanine aminotransferase; AST = aspartate aminotransferase; SEM = standard error of the mean. ^{a} Tissue volume for the two infusion methods were then compared using a t-test.

The results of this study further reinforce the limitations of direct injection. Therapeutic injected within the liver displayed relatively limited diffusion within the tissue, often being confined to one to two 1 cm thick sections of liver. This distribution pattern would be insufficient to treat multifocal diffuse disease.

PEDD offers an alternative mode of therapeutic delivery. PEDD is conducted using a catheter system that is equipped with a one-way microvalve structure. When placed within the blood stream, the valve physically modulates blood flow and pressure. Forward blood flow is retained after device placement, allowing for the migration of therapeutics downstream into the target vascular network. During infusion, pressure can be generated locally within the arterial network without risk of reflux into non-target tissues.

In this study, normal porcine liver tissue was treated using a PEDD device with the goal of quantifying distribution and tissue uptake of the label ODN sequences. Using quantitative near-IR imaging of 1 cm slices of liver tissue, the full volume of tissue exposed to and retaining the labeled compound was quantified. The volume of tissue exposed to the ODN after PEDD infusion was over 7 times greater than that exposed by needle injection (99.0±28.8 cm³ SE vs. 13.5±2.9 cm³ respectively). Unexpectedly, the total luminous intensity, a measure of therapy uptake, was significantly greater in PEDD treated tissue as well (48997±10088 lu SE PEDD vs 19275±6352 lu SE needle injection, 2.5-fold increase).

Direct needle injection was expected to deliver the full volume of therapeutic into the targeted tissue, resulting in high delivery efficiency. However, significantly lower luminous intensity was observed relative to PEDD. This may be associated with the phenomena known as backflow, which results when the infusate migrates around the needle and flows out from the needle tract. Several factors influence the magnitude of this phenomenon, such as needle insertion rate, insertion angle, infusion rate, needle diameter, and tissue pressure. Backflow has been implicated in the reduction of efficacy of therapeutic injected into tumors. We hypothesize that a substantial portion of the infused ODN volume experienced backflow through the needle tract into the abdomen, resulting in lower tissue retention.

PEDD devices physically modulate blood flow and local vascular pressure, potentially contributing to the high levels of ODN retention observed in the study. At initial placement, the device induced a pressure gradient from 96.8 mmHg±6.2 mmHg SE proximal to the valve to 57.6 mmHg±8.3 mmHg SE distal to the valve (n=8 measurements). Reduction in pressure also results in slower blood flow velocities. During infusion, the therapeutic likely experienced greater contact time with the tissue, resulting in robust absorption.

The use of a PEDD device resulted in significantly more MAA deposition in tumor tissue while significantly reducing delivery to normal tissue relative to conventional catheter systems. The normal porcine liver treated in this study likely responded to the pressure gradient induced by the PEDD device resulting in some degree of flow redirection.

However, no tumor tissue was present in the current model, so differential flow to a diseased tissue state was not observed.

AST and ALT measurements were conducted over the course of 90 minutes post infusion of labeled ODN. This duration has been documented as being sufficient to discern significant elevations of enzyme levels after administration of known hepatotoxins such as carbon tetrachloride. While the dose of the labelled oligonucleotide administered in this study would be predicted to be subtherapeutic in preclinical models, these results further illustrate that the route of administration by PEDD did not result in significant elevations of liver enzymes. No signs of hepatotoxicity were observed over the course of the study.

In summary, the present study was designed to compare the delivery of near-IR labelled ODNs via conventional direct needle injection relative to infusion with PEDD technology using the TriNav® catheter system. The objective was to quantify the intensity of the signal and the volume of signal as indices of drug retention and tissue distribution. In this regard, drug delivery with PEDD/TriNav® significantly improved both signal intensity (an index of drug retention) and tissue volume exposed to drug (an index of drug distribution). This significant increase in therapeutic coverage could be a critical factor in treating diffuse disease. Such methodology may allow therapeutics to reach both macro and micro metastasis that would be otherwise impractical or impossible to treat using conventional needle injections.

### Example 5

The objective of this study was to evaluate to route of administration (via end-hole catheter versus local-regional PEDD infusion) on the intrahepatic localization of fluorescent-tagged Toll-like receptor 9 (TLR9) Class C agonists SD-101 (Cy5.5-SD-101 and IRDye800CW-SD-101).

After delivery of the investigative compounds, hepatic tissues were analyzed using near InfraRed (near-IR) imaging to compare the two delivery modalities. Near-IR imaging utilizes a spectral window of relative tissue transparency and has been used extensively clinically for anatomical mapping and identification of cancerous tissues. Quantitative full organ analysis of therapeutic distribution and concentration was achieved.

### Materials and Methods

### Test Animals

The study was conducted on 8-11 week old female Yorkshire Cross swine ranging in weigh from 45-60 kg (Oak Hill Genetics, Ewing, IL).

### Oligonucleotide (ODN) Test Articles

The TLR9 agonist SD-101 sequence oligo was synthesized and conjugated to the Cy5.5 (ex. 685 nm, em. 706 nm) fluorophore.

The TLR9 agonist SD-101 sequence oligo was synthesized and conjugated to the IRDye800CW (ex. 767 nm, em. 791) fluorophore.

### Study Design

A total of 8 healthy female swine were selected for the study. All animals received two infusions of therapeutic, with the first infusion conducted using an end-hole catheter followed by a second infusion with a PEDD device (TriNav®, Cat No TNV-21120-35, TriSalus Life Science, Westminster CO) 15 minutes after completion of the first infusion. The order of test article infusion was alternated, with four animals each receiving infusion with Cy5.5-SD-101 or IRD800CW-SD-101 through the end-hole catheter for the first infusion and four animals each receiving infusion with Cy5.5-SD-101 or IRD800CW-SD-101 through the PEDD catheter for the second infusion.

The device order was not switched as part of this study design. This is due to possible short term (15-30 min) alterations in vessel physiology resulting from the PEDD infusion confounding the hemodynamics pattern associated with the end-hole catheter and to minimize the chance of vascular spasm preventing a second infusion from taking place in a timely manner.

### Test Article Preparation

The lyophilized test article pellets were resuspended in pure DNase free water at a concentration of 2.5 nmol/ml. The ODN concentrate was then aliquoted into 1 cc tubes and stored at -60 to -80° C. prior of use.

At the time of administration, the respective frozen ODN concentrate was thawed. The ODN concentrate was diluted with 9 mL of sterile saline solution to produce a 10 mL total volume containing 2.5 nmol of compound and stored in a 10 mL polypropylene syringe (BD Bioscience, San Jose, CA) prior to infusion. Dosage was chosen to provide optimal imaging signal and is predicted to be subtherapeutic. Syringes containing the ODN were imaged using the Pearl Trilogy Imaging System (Li-Cor, Lincoln NE) to quantify the initial luminous intensity of the infusate prior to administration and ensure consistency in dosage between sample groups.

### Local Hepatic Arterial Infusion

The Seldinger technique was used to gain access through the femoral artery. A 5F introducer sheath (Pinnacle, Terumo Medical Corporation, Somerset, NJ) was secured at the site. A 5F angiographic catheter (Glidecath, Terumo Medical Corporation, Somerset, NJ) was used to perform angiography to identify hepatic arterial anatomy. A 1.5- to 3.5-mm diameter vessel (mean 2.98 mm±0.13 mm SE) feeding the left medial and/or left lateral lobe was then selected. The end-hole catheter was then tracked to the target location (various, 0.018”-0.021” ID, (Direxion, Rebar-18, Renegade, Boston Scientific, Marlborough, MA), (Excelsior 1018, Stryker Neurovascular, Fremont CA), and (Transit, Cordis, Miami Lakes, FL)).

An invasive blood pressure (IBP) transducer (TruWave PX600, Edwards Lifesciences Corp., Irvine CA) connected to a Quantien pressure Monitor (Abbott. Abbott Park, IL) was used to measure pressure through the lumen of the infusion system (at distal tip) and through the lumen of the angiographic catheter (proximal to the infusion lumen). The 10 mL therapeutic syringe was then placed into a syringe pump (NE-1000, New Era Pump Systems Inc. Farmingdale, NY) and the first test article solution was infused through an end-hole catheter at a rate of 2 mL/min for a total duration of 5 minutes. This rate and volume are consistent with clinical practice for HA infusion. After infusion, the device was allowed to remain in position for 5 minutes and was then withdrawn.

The TriNav® infusion system was then tracked into the exact same location within the target vessel as confirmed by angiography. The second infusion was conducted in an identical manner with the alternative color channel ODN. The PEDD was allowed to remain in position for 5 minutes after the completion of infusion.

### Tissue Preparation

Animals were then sacrificed after device removal and the liver was removed. Each lobe of the liver was separated and near-IR imaging with a Pearl Trilogy Imaging System was performed to identify patterns of drug uptake. Each lobe was then cut into 1 cm thick sections, as described in Example 4 (FIG. 12) and separately imaged (85 µm resolution, 700 nm, 800 nm, and white light channels) on both tissue faces using the Pearl system. The full liver volume was analyzed for the presence of the ODN tool compound within the tissue.

### Therapeutic Signal Quantification

Therapeutic signal quantification was conducted according to the process described in Example 4 (FIGS. 13A-13B). Overlap of tissue treated within the infusion zone was assessed by assigning a coordinate for each pixel within the image. Pixels displaying signal above the “high” threshold for both the red and green channel were considered areas where both treatments overlapped. Pixels were quantified for areas of green channel signal presence, red channel signal presence, and overlapping green and red signal presence.

### Results

### Test Article Tissue Uptake

Near-IR imaging of liver was used to quantify the signal intensity and distribution of the absorbed ODN test articles delivered by the end-hole and PEDD devices (n=4 per device / labeled ODN). Data was analyzed using a 2-sample students t-test in MiniTab Software (Minitab LLC, Chicago, IL). Both ODN articles displayed the same trend of increasing signal when delivered by PEDD (Table 3). Pooling the signal intensity measurements for both ODN articles (n=8 per device) demonstrated a significant increase in signal intensity when delivered by PEDD (FIG. 1. p=0.033).

TABLE 3

Signal Intensity (lu)

| ODN | End-hole Catheter Mean lu (SE) | PEDD Infusion System Mean lu (SE) | p-Value |
|---|---|---|---|
| IRD800CW-SD-101 (n=4) | 77962 (22656) | 91211 (7522) | 0.348 |
| Cy5.5-SD-101 (n=4) | 33252 (20729) | 85272 (33045) | 0.045 |
| Combined (n=8) | 55607 (31230) | 88242 (22413) | 0.033 |

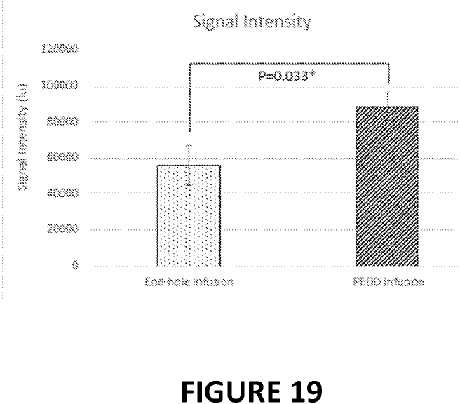

FIG. 19 represents signal intensity of labeled ODN retained in liver tissue after delivery by an end-hole catheter or by PEDD.

In addition, no significant differences in tissue coverage were observed when treating the normal porcine liver with an end-hole device vs a PEDD device (Table 4).

TABLE 4

Treated Tissue Volume (cm²)

| ODN | End-hole Catheter Mean lu (SE) | PEDD Infusion System Mean lu (SE) | p-Value |
|---|---|---|---|
| IRD800CW-SD-101 (n=4) | 185.9 (70.2) | 172.2 (48.5) | 0.762 |
| Cy5.5-SD-101 (n=4) | 63.2 (35.8) | 101.1 (45.6) | 0.249 |
| Combined (n=8) | 124.5 (83.4) | 136.6 (57.9) | 0.742 |

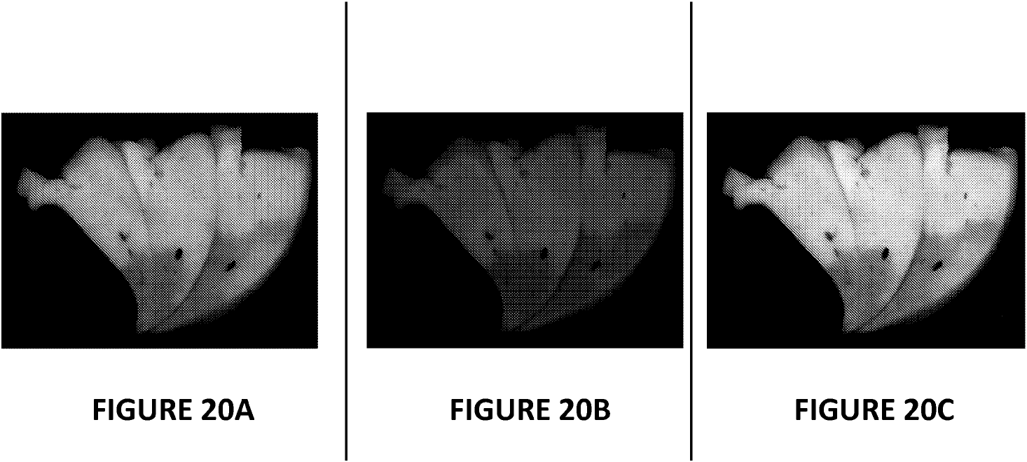

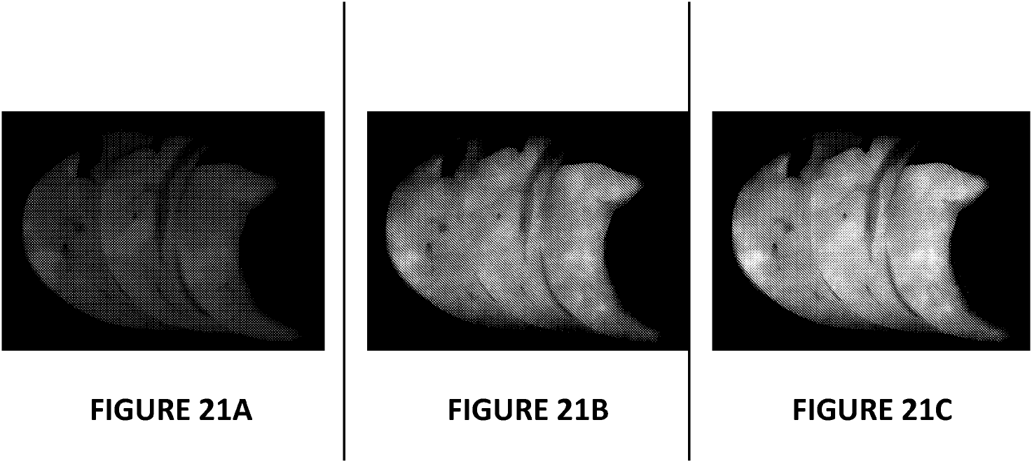

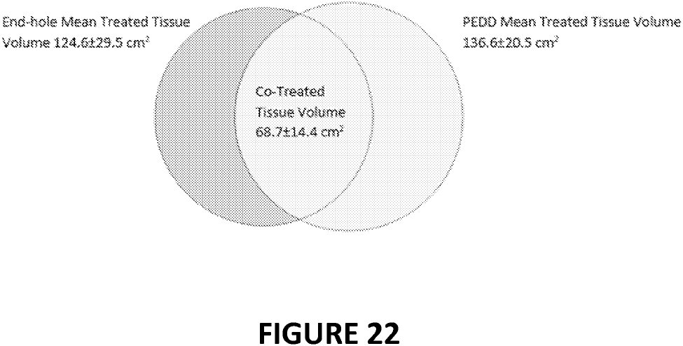

While delivery device did not significantly impact the overall volume of treated tissue, the distribution of the labeled ODN did not fully coincide between infusions (Table 5). Some animals displayed a high degree of homology in distribution pattern between the two infusions (FIGS. 20A-20C) while other samples displayed a heterogeneous distribution (FIGS. 21A-21C). For example, FIGS. 20A-20C illustrate tissue displaying a high degree of signal overlap from infusion with end-hole and PEDD devices: (a) FIG. 20A shows 800 nm green channel distribution of IRD800CW-SD-101 delivered using an end-hole catheter (b) FIG. 20B shows 700 nm red channel distribution of Cy5.5-SD-101 delivered by PEDD, and (c) FIG. 20C shows composite image illustrating overlap of distribution with the infusion devices. FIGS. 21A-20C illustrate tissue displaying a low degree of signal overlap from infusion with end-hole and PEDD devices: (a) FIG. 21A shows 700 nm red channel distribution of Cy5.5-SD-101 delivered using an end-hole catheter (b) FIG. 21B shows 800 nm green channel distribution of IRD800CW-SD-101 delivered by PEDD, and (c) FIG. 21C shows composite image illustrating overlap of distribution with the infusion devices. FIG. 22 illustrates a Venn diagram comparing end-hole mean treated tissue volume, PEDD mean treated tissue volume, and overlapping co-treated tissue volume.

TABLE 5

Tissue Distribution of Labeled ODN

| Subject ID | End-hole Infusion Volume (cm²) | PEDD Infusion Volume (cm²) | Overlapping Volume (cm²) | Percent of End-hole Volume overlapping PEDD infusion Zone |
|---|---|---|---|---|
| P20112 | 93.7 | 166.5 | 75.9 | 45.6% |
| P20126 | 91.3 | 153.5 | 54.3 | 35.4% |
| P20127 | 152.7 | 82.8 | 69.3 | 83.7% |
| P21055 | 166.7 | 125.0 | 115.0 | 92.0% |
| P21094 | 48.2 | 240.9 | 39.6 | 16.4% |
| P21095 | 289.3 | 149.9 | 139.0 | 92.8% |
| P21109 | 19.7 | 128.0 | 17.8 | 13.9% |
| P21111 | 134.8 | 46.7 | 39.5 | 84.5% |
| Mean (SE) | 136.6 (20.5) | 124.6 (29.5) | 68.7 (14.4) | 58.0 (12)% |

### Pressure Modulation

The expandable microvalve equipped on the TriNav® device acts to modulate local vascular pressure. The one way valve generates a local pressure gradient with higher pressure present proximal to the valve relative to that distal to the valve. Mean pressure proximal to the valve (measured through the base catheter) was 87±4 mmHg while distal vascular pressure was significantly lower at 50±7 mmHg (p=0.0001), representing a 43% reduction in vascular pressure. The end-hole did not significantly impact vascular pressure with proximal pressure measured as 93±6 mmHg and distal pressure of 88±9 mmHg.

It should be noted that device order was not altered in this study, with the end-hole infusion occurring first and the PEDD infusion occurring second in order. A 5-minute period was specified to allow any labeled ODN present in the blood to circulate though the organ, after which the PEDD device was positioned within the vessel for the second infusion. This procedure typically resulted in both infusions being conducted within 15 minutes of each other. It is possible that the longer circulation period may have washed some labeled SD-101 from the tissue, resulting in higher signal intensity for the PEDD infusion relative to the end-hole infusion. However, this effect would be anticipated to occur over a longer time period than the rapid washout of material from within the blood vessels, which was accounted for in the washout period in the experimental design. In previous research, a TriNav® device was used to infuse Cy5.5-SD-101 in an identical manner (n=4), however blood was allowed to circulate for a minimum of 90 minutes prior to organ analysis. There was no significant difference in the signal intensity for subjects experiencing a 90-minute long circulation period compared to the 5 minute period experienced by subjects in this study (51,511±21267 lu SE vs 85,272±16,523 respectively (p=0.265)).

### Conclusions

The present study was designed to compare the delivery of near-IR labelled ODNs via conventional end-hole catheter infusion relative to infusion with PEDD technology using the TriNav® catheter system. The objective was to quantify the intensity of the signal and the volume of signal as indices of drug retention and tissue distribution.

Drug delivery with PEDD/TriNav® significantly increased signal intensity (an index of drug retention) when compared to the end-hole device. This may be associated with the modulation of pressure and flow within the vasculature induced by placement of the TriNav®, resulting in higher tissue absorption of the test articles. Tissue coverage patterns between the end-hole catheter and the TriNav® also diverged despite infusion in identical locations. Blood flow is sensitive to changes in local pressure and the position of the infusion lumen within a vessel. The microvalve attached to the TriNav® device tends to self-center the infusion lumen of the device within the vessel while generating a local decrease in blood pressure and flow. These physical characteristics of the TriNav® device likely resulted in the redistribution of blood flow and associated therapeutic deposition observed in this study.

### Example 6

In this example, liver-directed infusions of a TLR9 agonist, SD-101, were administered with the aim of enhancing response rates to CPI therapy with stage 4 UM and to improve UM metastatic tumors, e.g. liver metastases (LM). In this regard, the SD-101 can be used for the treatment of all UM LM lesions. Further, through the distal effects of SD-101 in combination with CPI system infusions, extrahepatic lesions can benefit from enhanced immune-responsiveness as well.

The study has two phases, i.e. Phases 1 and 1b. In this regard, the primary objective for Phase 1 is to determine the safety of, and to identify, the maximum tolerated dose (MTD) or optimal dose of PEDD/HAI of SD-101 alone, the MTD or optimal dose of SD-101 in combination with nivolumab, and of SD-101 in combination with both ipilimumab and nivolumab. Further, the secondary objective is to determine whether single- or dual-agent CPI should be utilized in the Phase 1b portion of the study. With regard to Phase 1b, the primary objective is to assess the Response Evaluation Criteria in Solid Tumors (RECIST) v1.1 overall response rate (ORR) and 12-month overall success (OS) to PEDD/HAI SD-101 in combination with systemic, i.e. intravenous (IV), immunological checkpoint blockade (co-primary endpoints). Further, the secondary objective is to (i) assess efficacy in terms of RECIST for immunotherapy (iRECIST) ORR, mRECIST ORR, RECIST 1.1 hepatic-specific response rate (HRR), duration of response (DOR), overall progression-free survival PFS, and clinical benefit rate (complete response [CR] + partial response [PR] + SD) and (ii) assess the safety/toxicity of the chosen MTD or optimal dose of SD-101 in combination with CPI. Further, there are also exploratory objectives: (i) to assess RECIST v1.1 hepatic-specific progression-free survival (HPFS); (ii) to assess pathologic response in LM following PEDD/HAI of SD 101 with or without systemic IV CPI infusion and correlation with imaging response scoring; (iii) to assess liver tumor and plasma levels of SD 101 following PEDD/HAI; (iv) to assess the intratumoral immunological effects of treatment on myeloid-derived suppressor cells (MDSC), lymphocytes, and cytokine profiles using paired baseline and on-treatment liver tumor and normal liver biopsies; (v) to assess the peripheral immunological pharmacodynamic effects of treatment using serial blood sampling for CTC, circulating cytokines, and other immunologic correlatives; (vi) to assess abscopal effects of single-agent SD 101 via PEDD/HAI; (vii) to assess changes from baseline in ECOG PS over time; (viii) to assess changes from baseline in quality of life using the EORTC-QLQ-C30 instrument.

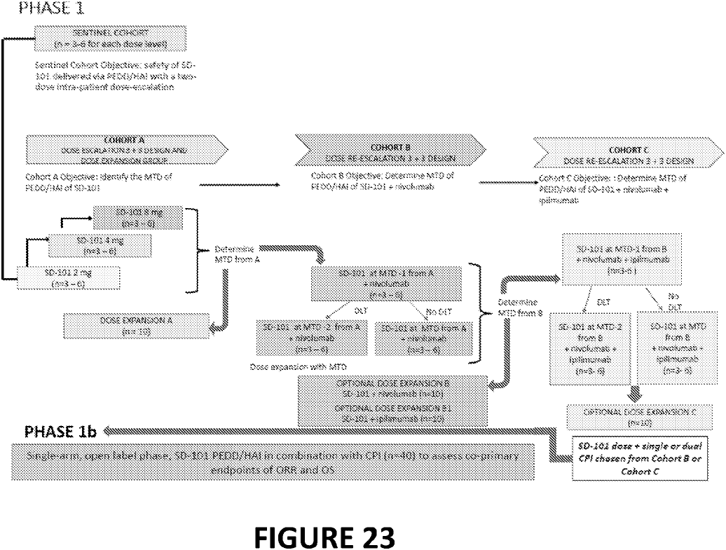

The overall design for the study can be found in FIG. 23. The study is open-label, multicenter, and nonrandomized.

As described in further detail below, in Phase 1, a Sentinel Cohort is enrolled to determine the safety of SD-101 delivered via PEDD/HAI with a two-dose intra-patient dose escalation. The Sentinel Cohort patients receive 2 infusions (2 weeks apart) whereby the first infusion comprises the first dose level (0.5 mg) and the second infusion comprises the second dose level (2 mg), with assessments for toxicity, prior to advancing to one of the cohorts of the trial. Patient enrollment in the Sentinel Cohort is staggered by 7 days. In the absence of dose-limiting toxicities (DLTs), each patient will be eligible to transition into Cohort A at the second infusion time point for dose level 1 (i.e. Cohort A, Day 8 dose). Following completion of the Sentinel Cohort, escalating doses of SD-101 are administered alone (Cohort A), together with nivolumab (Cohort B), and together with combined ipilimumab and nivolumab (Cohort C). Cohorts B and C begin one dose level below the MTD or optimal dose from Cohort A to optimize safety when adding CPI to SD-101. Cohort C begins after completion of Cohort B. A standard 3 + 3 dose-escalation design will be employed to determine the MTD.

Following determination of the MTD or optimal dose of SD-101 for PEDD/HAI and which CPI regimen(s) are tolerated, the approach progresses to Phase 1b. Patients in Phase 1b receive the SD-101 dose selected from Phase 1 in combination with single- or double-agent checkpoint blockade. The choice of single or dual CPI therapy in combination with SD-101 for Phase 1b can consider safety data in addition to response rates from Cohorts B and C in Phase 1. SD-101 is administered over two cycles, with three weekly doses per cycle for Phase 1 Cohorts A, B, and C, and Phase 1b. For the Sentinel Cohort, SD-101 will be administered over 1 mini cycle, consisting of two infusions delivered 2 weeks apart. Following the first SD-101 infusion for each patient, an overnight in-hospital observation or admission is required. If the first SD-101 dose is well tolerated, further overnight observation or admission is at the discretion of the treating physician for subsequent SD-101 infusions. If subsequent infusions are performed on an outpatient basis, the patient will be observed for a minimum of 6 hours post infusion before being discharged home, if clinically stable. If there are any Grade 2 events related to SD-101 PEDD/HAI that required inpatient therapy following the first infusion, the patient can be kept for overnight observation or admission following each subsequent SD-101 infusion.

### Inclusion Criteria

According to an embodiment, to be included in the study, the patient must meet all of the following criteria for inclusion:
1. Male or female, age ≥18 years of age at screening
2. Able to understand the study and provide written informed consent prior to any study procedures
3. Has histologically or cytologically confirmed metastatic UM with liver-only or liver-dominant disease. Liver-dominant disease is defined as:
   a. Phase 1, Cohort A - Intrahepatic metastases representing the largest fraction of disease relative to other organs, with permissible extrahepatic sites being the lungs, skin or subcutaneous tissues, and bone.
   b. Phase 1, Cohorts B and C and Phase 1b - Intrahepatic metastases representing the largest fraction of disease relative to other organs, or if progression of LM represent a significant threat to the patient’s life.
4. Has not received prior cytotoxic chemotherapy, targeted therapy, or external radiation therapy within 14 days prior to enrollment
5. Phase 1 only: Has not received therapy with prior immunological checkpoint blockade within 30 days before the first dose of study intervention and has no ongoing immune-mediated AEs Grade 2 or higher Phase 1b only: Has not ever received therapy with prior immunological checkpoint blockade
6. Has not ever received prior embolic HAI therapy with permanent embolic material. Note: Prior surgical resection or radiofrequency ablation of oligometastatic liver disease is allowed on both the Phase 1 and Phase 1b portions of this study. Liver lesions that received ablative therapies should not be considered target lesions unless they have clearly progressed since the therapy.
7. Has no prior history of or other concurrent malignancy unless the malignancy is clinically insignificant, no ongoing treatment is required, and the patient is clinically stable
8. Has measurable disease in the liver according to RECIST v.1.1 criteria
9. Has an ECOG PS of 0-1 at screening
10. Has a life expectancy of >3 months at screening as estimated by the investigator
11. Has a QTc interval ≤480 msec
12. All associated clinically significant (in the judgment of the investigator) drug-related toxicity from previous cancer therapy must be resolved (to Grade ≤1 or the patient’s pretreatment level) prior to study treatment administration (Grade 2 alopecia and endocrinopathies controlled on replacement therapy are allowed).
13. Has adequate organ function at screening as evidence by:
   Platelet count >100,000/µL
   Hemoglobin ≥8.0 g/dL
   White blood cell count (WBC) >2,000/µL
   Serum creatinine ≤2.0 mg/dL unless the measured creatinine clearance is >40 mL/min/1.73 m²
   Total and direct bilirubin ≤2.0 × the upper limit of normal (ULN) and alkaline phosphatase ≤5 × ULN. For patients with documented Gilbert’s disease, total bilirubin up to 3.0 mg/dL is allowed.
   ALT and AST ≤5 × ULN
   Prothrombin time/International Normalized Ratio (INR) or activated partial thromboplastin time (aPTT) test results at screening ≤1.5 × ULN (this applies only to patients who do not receive therapeutic anticoagulation; patients receiving therapeutic anticoagulation should be on a stable dose for at least 4 weeks prior to the first dose of study intervention)
   Note: Laboratory tests with exclusionary results judged by the investigator as not compatible with the patient’s clinical status may be repeated once for eligibility purposes.
14. Females of childbearing potential must be nonpregnant and nonlactating, or postmenopausal, and have a negative serum human chorionic gonadotropin (hCG) pregnancy test result at screening and prior to the first dose of study intervention.
   Females of childbearing potential must agree to abstain from sexual activity with male partners, or if sexually active with a nonsterilized male partner must agree to use highly effective methods of contraception from screening, throughout the study and agree to continue using such precautions for 100 days after the final dose of study intervention.
   Nonsterilized males who are sexually active with a female of childbearing potential must agree to use effective methods of contraception and avoid sperm donation from

### Day 1 Throughout the Study and for 30 Days After the Final Dose of Study Intervention. Phase 1

The separate Phase 1 patient cohorts are as follows:

Sentinel Cohort - Intra-patient dose-escalation cohort of PEDD/HAI of SD-101 monotherapy using a standard 3 + 3 design followed by transition to Cohort A (see below) in the absence of DLTs:
Infusion #1 (Sentinel Day 1): 0.5 mg (n = 3-6)
Infusion #2 (Sentinel Day 15): 2 mg (n = 3-6)

Patient enrollment in the Sentinel Cohort is staggered by 7 days. A new patient is not be enrolled any sooner than 7 days following the completion of the first infusion of SD-101 in the prior patient.

Following Infusion #1 for each patient, a Safety Review Committee (SRC) can meet to review clinical data through Day 11 and determine whether dose escalation to Infusion #2 can occur. Following Infusion # 2 for each patient, the SRC can meet to review clinical data through Day 18, and in the absence of DLTs, and upon confirmation by the SRC, the patient is eligible to transition to Cohort A at the 2 mg dose level. If there is disagreement regarding interpretation of safety, the independent reviewer’s decision can determine the outcome.

Cohort A - Dose-escalation cohort of PEDD/HAI of SD-101 monotherapy (2 cycles, 1 dose per week × 3 weeks, per cycle) using a standard 3 + 3 design followed by an optional expansion group to identify the maximum tolerated dose (MTD) or optimal dose of SD-101 alone and estimate the monotherapy response rate:
Dose level 1: 2 mg (n = 3-6)
Dose level 2: 4 mg (n = 3-6)
Dose level 3: 8 mg (n = 3-6)

Enrollment of the first 2 patients at each dose level can be staggered by at least 48 hours. Further, assuming investigation of up to three dose levels of SD-101 (2, 4, and 8 mg), a minimum of 19 and a maximum of 28 patients can be used in Cohort A. Additional incremental dose levels (in increments of 4 mg) may be added based on safety and response data. An expansion group of ten patients at the SD-101 monotherapy MTD or optimal dose can proceed concurrently with Cohort B.

Cohort B - Standard 3 + 3 design dose re-escalation cohort of PEDD/HAI of SD-101 (2 cycles, 1 dose per week × 3 weeks, per cycle) in combination with intravenous (IV) nivolumab 480 mg every four weeks (Q4W) to identify the MTD or optimal dose of SD-101 with single-agent nivolumab:
Dose level 1: Given the potential for enhanced hepatotoxicity with this combination, the dose escalation for nivolumab in combination with PEDD/HAI of SD-101 begins at 1 dose level below the MTD or optimal dose from Cohort A (i.e. MTD-1 or optimal dose-1) (n = 3-6)
Dose level 2: Nivolumab in combination with PEDD/HAI of SD-101 at the MTD or optimal dose from Cohort A (n = 3-6), or if MTD-1 or optimal dose-1 not tolerated, can de-escalate to MTD-2 or optimal dose-2

Enrollment of the first 2 patients at each dose level can be staggered by at least 48 hours. Assuming investigation of up to 2 dose levels of SD-101 in combination with nivolumab in Cohort B, a minimum of 6 and a maximum of 12 patients can be required in Cohort B. An optional expansion group of up to ten patients receiving SD-101 in combination with nivolumab may proceed concurrently with Cohort C. An optional Cohort B1 of 10 patients to test single-agent ipilimumab in combination with PEDD/HAI of SD-101 at the MTD or optimal dose from Cohort A (n=3-6); or if MTD-1 or optimal dose-1 is not tolerated in Cohort B, Cohort B1 de-escalates to MTD-2 or optimal dose-2. Given that nivolumab and ipilimumab have different mechanisms of action (targeting PD-1 vs. CTLA-4), if biologic activity of nivolumab is low, study of single-agent ipilimumab may be desired before proceeding to Cohort C.

Cohort C - Standard 3 + 3 design dose re-escalation study of PEDD/HAI of SD-101 (2 cycles, 1 dose per week × 3 weeks, per cycle) in combination with systemic IV ipilimumab 3 mg/kg and systemic IV nivolumab 1 mg/kg every three weeks (Q3W) for four doses each followed thereafter by nivolumab 480 mg systemic IV Q4W to identify the MTD or optimal dose of SD-101 with dual-agent CPI:
Dose level 1: Ipilimumab and nivolumab in combination with PEDD/HAI of SD-101 at 1 dose level below the MTD or optimal dose from Cohort B (n = 3-6)
Dose level 2: Ipilimumab and nivolumab in combination with PEDD/HAI of SD-101 at the MTD or optimal dose of SD-101 from Cohort B (n = 3-6), or if MTD-1 or optimal dose-1 not tolerated, can de-escalate to MTD-2 or optimal dose-2.

Assuming investigation of up to 2 dose levels of SD-101 (first the MTD-1 or optimal dose-1, then MTD or optimal dose from Cohort B, or MTD-2/optimal dose-2 if MTD-⅟optimal dose-1 was not tolerated), a minimum of 6 and a maximum of 12 patients can be required in Cohort C. Enrollment of the first 2 patients at each dose level can be staggered by at least 48 hours. An optional expansion cohort of 10 patients may be enrolled if additional data are needed to decide between single- and dual-agent CPI in combination with SD-101 via PEDD/HAI for Phase 1b.

### Phase 1b

Phase 1b may proceed after completion of Cohort B or Cohort C and review of data by the SRC. This phase will be conducted according to the decision regarding the SD-101 dose and the CPI regimen. A two-stage design is used in Phase 1b to establish whether the proportion of responses at the SD-101 MTD or optimal dose + single- or dual-agent CPI is sufficiently high to warrant further testing.

The Phase 1b portion of the study contemplates up to 40 participants. A two-stage design with the smallest total sample (referred to as Minimax design; Simon 1989) is used. In the first stage, 22 patients will be enrolled, and if 3 or more responses are observed, the Phase 1b cohort will be expanded to a total of 40 patients to further assess efficacy. Enrollment in this cohort will be stopped if 2 or fewer responses are observed in the first stage. If the total number of responding is >7, the treatment will warrant further testing. An interim analysis can be performed after fifty patients have been treated to assess the overall response rate (ORR).

### Duration of Administration

### Duration of SD-101 Administration (All Participants in Phase 1 and Phase 1b):

Sentinel Cohort - Up to 7 doses of SD-101 (includes doses to be received after transitioning to Cohort A)

Cohorts A, B, C, and Phase 1b - Up to 6 doses (maximum of 2 cycles of SD-101, 3 weekly doses per cycle). Fewer doses or cycles of SD-101 may be administered on the basis of toxicity or tolerability. All patients receiving at least 1 PEDD/HAI dose of SD-101 will be considered evaluable.

### Duration of CPI Administration

Phase 1, Sentinel Cohort and Cohort A: not applicable
Phase 1, Cohort B and optional expansion cohort: up to twelve months of nivolumab 480 mg Q4W
Phase 1, optional Cohort B1: systemic IV ipilimumab 3 mg/kg Q3W for 4 doses
Phase 1, Cohort C and optional expansion cohort: (i) systemic IV nivolumab 1 mg/kg Q3W for 4 doses then 480 mg/kg Q4W for up to twelve months and (ii) systemic IV ipilimumab 3 mg/kg Q3W for 4 doses.
Phase 1b: CPI regimen as determined by Phase 1 data for up to twelve months

### Interventions Administered

The interventions and planned dose levels are summarized in Table 6 below.

TABLE 6

| | | | |
|---|---|---|---|
| Intervention Name | SD-101 | Opdivo® (nivolumab)^{a} | Yervoy® (ipilimumab)^{b} |
| Type | Drug (ODN) | Biologic (antibody) | Biologic (antibody) |
| Dose Formulation | Solution | Solution for injection | Solution for injection |
| Unit Dose Strength(s) | 13.4 mg/mL* | 40 mg/4 mL 100 mg/10 mL 240 mg/24 mL | 5 mg/mL |
| Dosage Level(s) | 0.5, 2, 4, or 8 mg ^{c} | 1 mg/kg IV 480 mg IV | 3 mg/kg IV |
| Route of Administration | Intravascular injection by PEDD/HAI over 30-60 minutes | IV | IV |
| Use | Experimental | Background intervention | Background intervention |
| IMP and NIMP | IMP | NIMP | NIMP |
| Sourcing | TriSalus | Commercial - site supply | Commercial -site supply |
| Packaging and Labeling | Single-use vial | Single-dose vials | Single-dose vials |

* Unit dose strength of SD-101 reflects only SD-101 oligo.

### Infusion of SD-101

The SD-101 solution is infused via the hepatic arterial system, optionally using TriNav® Infusion System. Femoral or brachial/radial access may be used. Hemangiomata, shunting vessels, or other vascular lesions in the liver that may interfere with therapeutic delivery are embolized at the discretion of the treating interventional radiology specialist. For the SD-101 infusion procedures, the drug is prepared and delivered in a 50-mL syringe (therapeutic dose) and 100-mL vial containing the volume necessary for the therapeutic flush (10 mL), both at the therapeutic concentration, which can be provided as described in Table 7 below.

TABLE 7

| Cohort | SD-101 Dose to be Administered (mg) | Volume to be Administered (mL) | Dose SD-101 Concentration (mg/mL) | Total Volume Required | mg Sd-101 per required volume | Volume SD-101 Stock Solution | Volume Saline solution | Number of DP Vials Required |
|---|---|---|---|---|---|---|---|---|
| 1 | 2 mg | 50.0 mL | 0.04 mg/ml | 80 ml | 3.2 | 0.24 ml | 80 ml | 1 Vial, 13.4 mg/mL* * |
| 2 | 4 mg | 50.0 mL | 0.08 mg/ml | 80 ml | 6.4 | 0.48 ml | 80 ml | 1 Vial, 13.4 mg/mL* * |
| 3 | 8 mg | 50.0 mL | 0.16 mg/ml | 80 ml | 12.8 | 0.96 ml | 80 ml | 2 Vials, 13.4 mg/mL ** |
| 4* | 1 mg | 50.0 mL | 0.02 mg/ml | 80 ml | 1.6 | 0.12 ml | 80 ml | 1 Vial, 13.4 mg/mL* * |

*May be added based on safety and response data
** Unit dsose strength of SD-101 (13.4 mg/mL) reflects only SD-101 oligo.

According to an embodiment, a procedure workflow for the SD-101 treatment sessions can include the following: (i) gaining access to patient vasculature via the Seldinger technique through the femoral artery; (ii) preparing a heparinized saline flush (unless contraindicated for the patient) using a pressure bag; (iii) attaching the heparinized saline line to the base catheter and ensuring continuous flush throughout the procedure; (iv) connecting an Invasive Blood Pressure Transducer (IBP) to a patient monitor; (v) tracking the device to the target treatment location; (vi) attaching a high-pressure tubing line and flushing the transducer and line, ensuring no bubbles are present; (vii) attaching a high-pressure tubing line to the hub of the TriNav® Infusion System; (viii) allowing pressure reading to stabilize and recording the mean distal vascular pressure; (ix) disconnecting the high-pressure tubing line from the TriNav® Infusion System, and maintaining the line in the sterile field for subsequent pressure measurements; (x) connecting the 10-mL priming syringe to the stopcock, and flushing until the high-pressure tubing is completely filled with therapeutic and solution exits the end of the tubing; (xi) connecting the 50-mL syringe to the high-pressure tubing; (xii) placing the 50-mL syringe into the syringe pump; (xiii) prior to infusion, flushing the distal end of the stopcock with the 10-mL priming syringe until fluid exits the stopcock; (xiv) connecting the hub of the TriNav® Infusion System to the stopcock, and rotating the stopcock into position to infuse from the 50-mL therapeutic syringe; (xv) exchanging the 10-mL priming syringe for a 1-mL saline bolus syringe; (xvi) infusing, with the syringe pump, the calculated volume of the therapeutic (50-mL/# of sites) from the 50-mL syringe for 2 minutes at a rate of 2-mL/min (e.g. 4 mL volume); (xvii) rotating stopcock to the 1-mL syringe, and infusing the 1-mL saline aliquot by hand at a rate of 2-mL/sec (e.g. 0.5 sec duration), thereby generating positive pressure in a hepatic arterial infusion and promoting uptake of the therapeutic into the tumor; (xviii) after bolus infusion, rotating stopcock to continue infusion from the 50-mL syringe; (xix) infusing the remaining calculated volume of therapeutic at a rate of 2-mL/min; (xx) after completing infusion of the desired volume (displayed on pump), halting further infusion and disconnecting the high-pressure tubing from the TriNav® Infusion System; (xxi) clearing the registered volume delivered in preparation for the following infusion; and (xxii) repeating steps (iv) to (xxi) for remaining selective infusions.

In one embodiment, the 50.0 mL volume to be administered is allocated by per segment or sector of the liver. In an embodiment, calculation of infused volume per segment or sector may be performed prior to the SD-101 infusion procedure (based on pre-procedure MRI or CT scan), or during the same session as the SD-101 infusion (based on the angiography described above). In one embodiment, the 50-mL therapeutic dose can be allocated as follows: 3 × 10 mL infusions into target vessels in the right hepatic lobe and 2 × 10 mL infusions into target vessels in the left hepatic lobe. Further, the distribution of the 10-mL aliquots may be adjusted based on the location of measurable disease and target vessel diameter. In another embodiment, infused volume per segment or sector is calculated as follows: (perfused liver volume/total liver volume) * 40 + (estimated tumor volume in perfused segment/estimated total tumor volume in liver) * 10). In one embodiment, the planned SD-101 volume per segment or sector that was determined for the initial SD- 101 infusion procedure should be used as the planned volume for each subsequent SD-101 infusion procedure.

### Tumor Response Evaluations

All patients can undergo imaging with magnetic resonance imaging (MRI) and positron emission tomography (PET)/computed tomography (CT) to assess extent of disease in the liver and other sites, as well as liver biopsies and assays of circulating tumor cells (CTC), and circulating cytokines, and other immunologic correlatives. Tumor response can be measured radiographically using standard Response Evaluation Criteria in Solid Tumors (RECIST) v1.1 criteria. Official response scoring (RECIST v1.1) is assessable by Day 84. LM response is assessed on abdominal CT or MRI, while extrahepatic lesions will be assessed on whole-body PET/CT scans. Final response scoring is determined on Day 168 to ensure that pseudoprogression is ruled out and that initial response is confirmed. Imaging procedures will occur every 90 days thereafter. Hepatic imaging using MRI with Eovist® contrast should be used whenever possible for assessment

Four liver biopsies can be performed:
Phase 1 Sentinel Cohort - an initial biopsy is obtained on Day 15 before the 2 mg infusion of SD-101, and post infusion on Day 15 (for SD-101 levels in tumor tissue)
Phase 1 Cohorts A, B, and C, and Phase 1b: A baseline biopsy is obtained on Day 1 before the first infusion of SD-101 and post-infusion on Day 1 (for SD-101 levels in tumor tissue), at the beginning of the second cycle of SD-101 (before SD-101 Infusion #4), and at Day 100. Pathologic response can be assessed based on review by the local site pathologist with scoring of necrosis and fibrosis within tumor samples.

### Pharmacokinetics

Blood samples can be collected to characterize SD-101 systemic exposure after PEDD/HAI. No sampling or testing can be done for nivolumab or ipilimumab concentrations.

Tumor levels of SD-101 can be measured in the post-infusion biopsy specimens of LM obtained for tumor response assessments and from additional post-infusion biopsies on Day 15 for the Sentinel Cohort and Day 1 for Cohorts A, B, and C.

### Pharmacodynamics

Blood samples can be collected for the measurement of CTC, circulating cytokines, and other immunologic correlatives including IFN-α and IFN-γ related gene signatures, which may be more informative than pharmacokinetic assessments for this class of therapeutic.

### Safety

Safety assessments include adverse events (AEs), clinical laboratory testing, vital signs, physical examinations, and electrocardiograms (ECGs).

The following are considered DLTs when observed during either SD-101 cycle or within 4 weeks after the last SD-101 dose in Cycle 1 and are considered attributable to study intervention (SD-101 or CPI therapy) and/or the PEDD device:
≥ Grade 4 cytokine release syndrome (CRS)
that does not recover to ≤ Grade 2 within 7 days
per National Cancer Institute (NCI) Common Terminology Criteria for Adverse Events (CTCAE)
Grade 3 CRS per NCI CTCAE that does not recover to ≤ Grade 2 within 7 days
autoimmune AE ≥ Grade 3 per NCI CTCAE
allergic reaction AEs ≥ Grade 3 per NCI CTCAE
Grade 4 hematologic AEs that do not recover to ≤ Grade 2 within 7 days
Any Grade 4 AE per NCI CTCAE in any organ system

Patients who develop a DLT during either cycle of SD-101 can be permanently discontinued from study interventions unless adequate justification is provided that an alternative approach (e.g. as clinically indicated with dose modification) is expected to be reasonably safe given the specific DLT. The patient can be treated according to clinical practice and monitored for resolution of the toxicity.

SD-101 and/or CPI therapy can be permanently discontinued for severe or life-threatening infusion-related reactions. Dose interruptions, delays, or discontinuation of SD-101 and/or CPI therapy is required when a patient has a Grade 3 or higher immune-mediated reaction. Discontinuation of SD-101 and/or CPI therapy for abnormal liver tests is required when a patient meets one of the conditions outlined below or in the presence of abnormal liver chemistries not meeting protocol-specified stopping rules if the investigator believes that it is in best interest of the patient.
Patient is clinically jaundiced
Patient has evidence of coagulopathy
Patient has clinical evidence of portal hypertension including but not limited to ascites or variceal bleeding

All patients can be followed in this study for safety for at least 1 year after the start of treatment. All patients enrolled in the study may be assessed for overall survival (OS) beyond 1 year in a separate long-term follow-up protocol.

Patients who discontinue study treatment for reasons other than disease progression (e.g. toxicity, withdrawal of consent) can continue to undergo scheduled tumor assessments every 90 days until the patient dies, experiences disease progression (intra- or extrahepatic), or initiates further systemic cancer therapy, whichever occurs first.

### Rescue Medication and Therapy

The study site will supply immunomodulatory rescue medication that will be obtained locally. The following rescue medications may be used:

### Medications

1. IL 6 receptor antibody Tocilizumab 4-8 mg/kg systemic IV over 60 minutes for CRS, may repeat as clinically indicated
2. Methylprednisolone 2 mg/kg systemic IV bolus followed by 0.5 mg/kg IV every 6-12 hours for CRS Grade >2, or neurologic dysfunction. The first dose can be given without consultation with the investigator or designee; however, the subsequent doses must be given after the consultation with the study investigator or designee.
3. Consider 1 to 2 doses of anti-TNFα agents (infliximab or etanercept). The benefits are not known, but since TNFα may rise acutely, it is worth considering early in the disease process.
4. Etanercept 25 mg SC twice a week for 2 doses, given 3 to 4 days apart (0.4 mg/kg twice a week, maximum of 25 mg per dose
5. Infliximab dose 10 mg/kg systemic IV weekly × 2 doses

### Interventions

1. Endoscopic cholangiography and stent placement
2. Percutaneous cholangiography and stent placement

Although the use of rescue medications is allowable at any time during the study, the use of rescue medications should be delayed, if possible and clinically appropriate, for at least 6 hours following the administration of study intervention. The date and time of rescue medication administration, as well as the name and dosage regimen of the rescue medication must be recorded.

Adverse events clearly due to disease progression, unrelated to study drug, or expected for the eligible patient population of the study are not considered DLTs.

### Assessment for Cytokine Release Syndrome

The Investigator, or designee, will assess each patient for the presence of severe CRS. CRS grading will be determined based on the NCI CTCAE v5.0.

### Imaging

Extent of disease will be measured radiographically at the time points for the Sentinel Cohort, Cohort A, Cohort B, and Cohort C. Screening assessments must include an MRI of the abdomen and pelvis (with oral/systemic IV Eovist contrast unless contraindicated) and a brain scan (CT with systemic IV contrast or MRI). A spiral CT scan of the chest should be obtained in addition to the MRI of the abdomen and pelvis. If an MRI is medically contraindicated or at the discretion of the physician, a CT scan of the chest, abdomen, and pelvis may be performed using triple phase systemic IV contrast. If a PET/CT scan is performed, the CT portion of the study must be consistent with the standards for a full-contrast CT scan. Hepatic imaging using MRI with Eovist contrast should be used whenever possible for assessment of LM, along with PET/CT for assessment of disease outside of the liver. The same imaging method used at screening must be used throughout the study.

Any evaluable or measurable disease must be documented at screening and reassessed at each subsequent tumor evaluation. For patients with measurable disease, response will be assessed per RECIST v1.1. Local imaging reads will be utilized for response assessment during Phase 1. Independent Central Review (ICR) for response assessment may be performed during Phase 1b.

At the investigator’s discretion, imaging may be performed at any time if PD is suspected. In addition, mRECIST and iRECIST assessments will be performed for secondary endpoint data collection but will not be incorporated into official response scoring.

### ECOG Performance Status

The ECOG PS scale will be used to assess how the disease is affecting the patient’s daily living activities and ability to take care of themselves. At each specified time point, qualified site personnel will rate the patient according to the following scale:
0. Fully active, able to carry on all predisease performance without restriction
1. Restricted in physically strenuous activity but ambulatory and able to carry out work of a light or sedentary nature, e.g. light house work, office work
2. Ambulatory and capable of all self-care but unable to carry out any work activities; up and about more than 50% of waking hours
3. Capable of only limited self-care; confined to bed or chair more than 50% of waking hours
4. Completely disabled; cannot carry on any self-care; totally confined to bed or chair
5. Dead

Changes, i.e. worsening, will not be documented as AEs unless reported during the nondirected questioning for AEs.

### RECIST V1.1 Definitions

Measurable disease - The presence of at least 1 measurable lesion. If the measurable disease is restricted to a solitary lesion, its neoplastic nature should be confirmed by cytology/histology.

Measurable lesions - Lesion that can be accurately measured in at least 1 dimension with longest diameter ≥10 mm (CT scan slice thickness ≤5 mm).

Nonmeasurable lesions - All other lesions, including small lesions (longest diameter <10 mm), as well as truly nonmeasurable lesions (such as leptomeningeal disease, ascites, pleural/pericardial effusion, inflammatory breast disease, lymphangitic involvement of skin or lung, abdominal masses that are not measurable by reproducible imaging techniques).

### Baseline Documentation

All measurable lesions up to a maximum of 2 lesions per organ and 5 lesions in total, representative of all involved organs should be identified as target lesions and recorded and measured at baseline.

Target lesions should be selected on the basis of their size (lesions with the longest diameter) and their suitability for accurate repeated measurements by consistent imaging techniques.

A sum of the longest diameter (LD) for all target lesions (non-nodal) will be calculated and reported as the baseline sum LD. The baseline sum LD will be used as reference by which to characterize the objective tumor response in the measurable dimension of the disease.

All other lesions (or sites of disease) should be identified as nontarget lesions and should also be recorded at baseline. Measurements of these lesions are not required, but the presence or absence of each should be noted throughout follow-up.

### Evaluation of Target Lesions

### Complete Response (CR): Disappearance of All Target Lesions

Partial Response (PR): At least a 30% decrease in the sum of the LD of target lesions, taking as reference the baseline sum LD.

Progressive Disease (PD): At least a 20% increase in the sum of the LD of target lesions, taking as reference the smallest sum LD recorded since the treatment started or the appearance of 1 or more new lesions. The sum must demonstrate an absolute increase on 5 mm.

Stable Disease (SD): Neither sufficient shrinkage to qualify for PR nor sufficient increase to qualify for PD, taking as reference the smallest sum LD since the treatment started.

### Evaluation of Nontarget Lesions

Complete Response (CR): Disappearance of all nontarget lesions
Non-CR/Non-PD: Persistence of one or more nontarget lesion(s)
Progressive Disease (PD): Unequivocal progression of existing nontarget lesions, and/or the appearance of one or more new lesions.

Overall Survival in terms of Complete Response, Partial Response, and Stable Disease in RECIST 1.1 is assessed according to the following:

TABLE 8

| | | | |
|---|---|---|---|
| Target Lesions | Non-target Lesions | New Lesions | Overall Response |
| Complete Response | Complete Response | No | Complete Response |
| Complete Response | Non-Complete Response / Non- | No | Partial Response |
| | Progressive Disease | | |
| Partial Response | Non-Progressive Disease | No | Partial Response |
| Stable Disease | Non-Progressive Disease | No | Stable Disease |
| Progressive Disease | Any | Yes or No | Progressive Disease |
| Any | Progressive Disease | Yes or No | Progressive Disease |
| Any | Any | Yes | Progressive Disease |

### Duration of Overall Response

The duration of overall response is measured from the time measurement criteria are met for CR or PR (whichever is first recorded) until the first date that recurrent or PD is objectively documented (taking as reference for PD the smallest measurements recorded since the treatment started). The duration of overall CR is measured from the time measurement criteria are first met for CR until the first date that PD is objectively documented. Duration of SD: Stable disease is measured from the start of the treatment until the criteria for progression are met, taking as reference the smallest sum of measurements recorded since the treatment started, including the baseline measurements.

### Overall Survival

For all patients, OS will be calculated from the date of enrollment to the time of death. Patients who are still alive prior to the data cutoff for final efficacy analysis, or who dropout prior to study end, will be censored at the day they were last known to be alive.

### Progression Free Survival

For all patients, PFS will be calculated from the date of enrollment to the time of CT scan documenting relapse (or other unambiguous indicator of disease development), or date of death, whichever occurs first. Patients who have no documented relapse and are still alive prior to the data cutoff for final efficacy analysis, or who dropout prior to study end, will be censored at the date of the last radiological evidence documenting absence of relapse.

### Modified RECIST (mRECIST)

mRECIST definitions for hepatocellular carcinoma are as follows:
Complete Response (CR) = Disappearance of any intratumoral arterial enhancement in all target lesions
Partial Response (PR) = At least a 30% decrease in the sum of diameters of viable (enhancement in the arterial phase) target lesions, taking as reference the baseline sum of the diameters of target lesions
Stable Disease (SD) = Any cases that do not qualify for either PR or progressive disease
Progressive Disease (PD) = An increase of at least 20% in the sum of the diameters of viable (enhancing) target lesions, taking as reference the smallest sum of the diameters of viable (enhancing) target lesions recorded since treatment started
Overall Survival in terms of Complete Response, Partial Response, and Stable Disease in mRECIST is assessed according to the following:

TABLE 9

| | | | |
|---|---|---|---|
| Target lesions | Nontarget lesions | New Lesions | Overall response |
| CR | CR | No | CR |
| CR | IR/SD | No | PR |
| PR | Non-PD | No | PR |
| SD | Non-PD | No | SD |
| PD | Any | Yes or No | PD |
| Any | PD | Yes or No | PD |
| Any | Any | Yes | PD |

Abbreviations: CR = complete response; PR = partial response; IR = incomplete response; SD = stable disease; PD = progressive disease.
Source: Lencioni R, Llovet JM. Modified RECIST (mRECIST) assessment for hepatocellular carcinoma. Sem Liver Dis. 2010;30:52-60.

### RECIST 1.1 for Immune Based Therapeutics (iRECIST)

Response will also be assessed by iRECIST. In brief, the main differences between RECIST and iRECIST are explained in Seymour et al 2017 as follows: “The principles used to determine objective tumor response are largely unchanged from RECIST 1.1, while a major change of iRECIST is the concept of ‘resetting the bar’ if RECIST 1.1 progression is followed at the next assessment by tumor shrinkage. iRECIST defines iUPD based on RECIST 1.1 principles; however iUPD requires confirmation; confirmation is based on observing either further increase in size (or in the number of new lesions) in the lesion category (i.e. target, nontarget disease) where progression was first identified, or progression (defined by RECIST 1.1) in lesion categories that had not previously met RECIST 1.1 progression criteria. If, however, progression is not confirmed as described above, but instead tumor shrinkage (compared to baseline) meeting the criteria of iCR, iPR or iSD, then the bar is reset so that iUPD must occur again (compared to nadir values) and then be confirmed (by further growth) at the next assessment for iCPD to be assigned. If there is no change in tumor size/extent from iUPD, then the time-point response would again be iUPD. This approach allows atypical responses, such as delayed responses that occur after pseudoprogression, to be identified, further understood and better characterized.”

TABLE 10

| Target lesions* | Nontarget lesions* | New Lesions* | Time-Point Response | |
|---|---|---|---|---|
| | | | No prior iUPD** | Prior iUPD**^{;}*** |
| iCR | iCR | No | iCR | iCR |
| iCR | Non-iCR/non-iUPD | No | iPR | iPR |
| iPR | Non-iCR/non-iUPD | No | iPR | iPR |
| iSD | Non-iCR/non-iUPD | No | iSD | iSD |
| iUPD with no change OR decrease from last TP | iUPD with no change OR decrease from last TP | Yes | NA | NLs confirms iCPD if NLs were previously identified and increase in size (≥5 mm in SOM for NLT or any increase for NLNT) or number. If no change in NLs (size or number) from last TP, remains iUPD |
| iSD | iUPD | No | iUPD | Remains iUPD unless iCPD confirmed based in further increase in size of NT disease (need not meet RECIST 1.1 criteria for unequivocal PD) |
| iUPD | Non-iCR/non-iUPD | No | iUPD | Remains iUPD unless iCPD confirmed based on: • further increase in SOM of at least 5 mm, otherwise remains iUPD |
| iUPD | iUPD | No | iUPD | Remains iUPD unless iCPD confirmed based on further increase in: • previously identified target lesion iUPD SOM ≥5 mm and / or • NT lesion iUPD (prior assessment - need not be unequivocal PD) |
| iUPD | iUPD | Yes | iUPD | Remains iUPD unless iCPD confirmed based on further increase in: • previously identified target lesion iUPD ≥5 mm and / or • previously identified NT lesion iUPD (need not be unequivocal) and /or • size or number of new lesions previously identified |
| Non-iUPD/PD | Non-iUPD/PD | Yes | iUPD | Remains iUPD unless iCPD confirmed based on: • increase in size or number of new lesions previously identified |

Abbreviations: iCPD = confirmed immune PD; iCR = immune complete response; iPR =immune partial response; iSD = immune stable disease; iUPD = unconfirmed immune PD; NL = new lesions; NT = nontarget; PD = progressive disease; SOM = sum of measures; TP = time point
* Using RECIST 1.1 principles. If no pseudoprogression occurs, RECIST 1.1 and iRECIST categories for CR, PR and SD would be the same. ** in any lesion category. *** previously identified in assessment immediately prior to this TP.
Source: Seymour L, Bogaerts J, Perrone A, Ford R, Schwartz LH, Mandrekar S, et al. iRECIST: guidelines for response criteria for use in trials testing immunotherapeutics. Lancet Oncol. 2017;18(3):E143-E152.

In this example, SD-101 was administered via HAI/PEDD to two human patients. The SD-101 was administered to the human patients with the TriNav® Infusion System. In this regard, the first patient was administered a dose of .5 mg of SD-101 and the second patient was administered a dose of 2.0 mg of SD-101. Biospecimens from the patients were obtained as followed:
Day 1 - Pre-infusion Cytokines, PK, ADA, Immunologic Correlatives
Day 1 - Post-infusion PK (15, 30 min, 1, 2, 4, 6 hours)
Day 8 - Cytokines, Immunologic Correlatives
Day 15 - Pre-infusion Cytokines, PK, Immunologic Correlatives
Day 15 - Post-infusion PK (15, 30 min, 1, 2, 4, 6 hours)
Day 15 - Pre-infusion Liver Biopsy
Day 15 - Post-infusion Liver Biopsy

### TS-PERIO-01 Treatment Summaries

Patient 101-001 has completed the Sentinel Cohort (0.5 mg and 2 mg SD-101 doses) and was the first patient to have received SD-101 via PEDD/HAI. In addition, pre-and post-SD-101 infusion liver biopsies were performed on Sentinel Day 15. Correlative blood specimens, including cytokines, immunologic correlatives including MDSC levels, PK, and ADA specimens have been collected at multiple time points. This patient has been cleared by the safety review committee to transition to Cohort A at the 2 mg dose. The patient has not experienced any SAEs related to SD-101 PEDD HAI.

Patient 101-002 has completed the Sentinel Cohort (0.5 mg and 2 mg SD-101 doses via PEDD/HAI). In addition, pre- and post-SD-101 infusion liver biopsies were performed on Sentinel Day 15. Correlative blood specimens, including cytokines, immunologic correlatives including MDSC levels, PK, and ADA specimens have been collected at multiple time points. A safety review committee meeting is scheduled to determine whether the patient can transition to Cohort A at the 2 mg dose. The patient has not experienced any SAEs related to SD-101.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for treating a liver metastasis of uveal melanoma comprising administering to a subject in need thereof a therapeutically effective amount of a toll-like receptor 9 agonist having the structure: 5′- TCG AAC GTT CGA ACG TTC GAA CGT TCG AAT-3′ (SEQ ID NO: 1).

2. The method of claim 1, wherein the TLR9 agonist is administered through a device by hepatic arterial infusion (HAI).

3. The method of claim 1, wherein the TLR9 agonist is administered through a device by portal vein infusion (PVI).

4. The method of claim 1, wherein the therapeutically effective amount of the TLR9 agonist is administered is selected from the group consisting of 0.5 mg, 1 mg, 2 mg, 4 mg, or 8 mg.

5. The method of claim 1, wherein the TLR9 agonist may be administered through a catheter device.

6. The method of claim 5, wherein the catheter device comprises a one-way valve that responds dynamically to local pressure changes.

7. The method of claim 5, wherein the TLR9 agonist is administered through the catheter device via pressure-enabled drug delivery.

8. The method of claim 7, wherein the TLR9 agonist is administered with a rate of infusion of about 1 cc/min to about 5 cc/min.

9. The method of claim 7, wherein the TLR9 agonist is administered for a period of time about 25 minutes.

10. The method of claim 1, wherein the TLR9 agonist is administered in combination with one or more checkpoint inhibitors, wherein the checkpoint inhibitors are administered systemically, either concurrently, before, or after the administration of the TLR9 agonist.

11. The method of claim 10, wherein the one or more checkpoint inhibitors include at least one of nivolumab, pembrolizumab, and cemiplimab, atezolizumab, avelumab, and durvalumab, and ipilimumab.

12. The method of claim 1, wherein the administration of the TLR9 agonist comprises a dosing regimen comprising cycles, in which one or more of the cycles comprise the administration of the TLR9 agonist via a catheter device by hepatic arterial infusion followed by the systemic administration of a checkpoint inhibitor.

13. The method of claim 10, wherein the one or more checkpoint inhibitors include at least one of nivolumab, pembrolizumab, and cemiplimab, atezolizumab, avelumab, and durvalumab, and ipilimumab.
